SEQUENCE LISTING (67 sequences)

SEQ ID NO: 1 - protein, 185 aa, Homo sapiens
MPRLFLFHLLEFCLLLNQFSRAVAAKWKDDVIKLCGRELVRAQIAICGMSTWSKRSLSQEDAPQTPRPVAEIVPSFINKDTETIIIMLEFIANLPPELKAALSERQPSLPELQQYVPALKDSNLSFEEFKKLIRNRQSEAADSNPSELKYLGLDTHSQKKRRPYVALFEKCCLIGCTKRSLAKYC

SEQ ID NO: 2 - protein, 23 aa, Homo sapiens
PYVALFEKCCLIGCTKRSLAKYC

SEQ ID NO: 3 - protein, 31 aa, Homo sapiens
VAAKWKDDVIKLCGRELVRAQIAICGMSTWS

SEQ ID NO: 4 - protein, 185 aa, Homo sapiens
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 5 - protein, 24 aa, Homo sapiens
QLYSALANKCCHVGCTKRSLARFC

SEQ ID NO: 6 - protein, 29 aa, Homo sapiens
DSWMEEVIKLCGRELVRAQIAICGMSTWS

SEQ ID NO: 7 - protein, 142 aa, Homo sapiens
MARYMLLLLLAVWVLTGELWPGAEARAAPYGVRLCGREFIRAVIFTCGGSRWRRSDILAHEAMGDTFPDADADEDSLAGELDEAMGSSEWLALTKSPQAFYRGRPSWQGTPGVLRGSRDVLAGLSSSCCKWGCSKSEISSLC

SEQ ID NO: 8 - protein, 24 aa, Homo sapiens
DVLAGLSSSCCKWGCSKSEISSLC

SEQ ID NO: 9 - protein, 27 aa, Homo sapiens
RAAPYGVRLCGREFIRAVIFTCGGSRW

SEQ ID NO: 10 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSS

SEQ ID NO: 11 - protein, 128 aa, synthetic construct
  REGION 1..128: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPP

SEQ ID NO: 12 - protein, 126 aa, synthetic construct
  REGION 1..126: note = Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSS

SEQ ID NO: 13 - protein, 128 aa, synthetic construct
  REGION 1..128: note = Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSPP

SEQ ID NO: 14 - protein, 5 aa, synthetic construct
  REGION 1..5: note = Synthetic Construct
GGGGQ

SEQ ID NO: 15 - protein, 4 aa, synthetic construct
  REGION 1..4: note = Synthetic Construct
GGGQ

SEQ ID NO: 16 - protein, 5 aa, synthetic construct
  REGION 1..5: note = Synthetic Construct
GGGGS

SEQ ID NO: 17 - protein, 4 aa, synthetic construct
  REGION 1..4: note = Synthetic Construct
PGPQ

SEQ ID NO: 18 - protein, 4 aa, synthetic construct
  REGION 1..4: note = Synthetic Construct
PGPA

SEQ ID NO: 19 - protein, 25 aa, synthetic construct
  REGION 1..25: note = Synthetic Construct
GGGGQGGGGQGGGGQGGGGQGGGGQ

SEQ ID NO: 20 - protein, 32 aa, synthetic construct
  REGION 1..32: note = Synthetic Construct
PGPQPGPQPGPQPGPQPGPQPGPQPGPQPGPQ

SEQ ID NO: 21 - protein, 32 aa, synthetic construct
  REGION 1..32: note = Synthetic Construct
PGPAPGPAPGPAPGPAPGPAPGPAPGPAPGPA

SEQ ID NO: 22 - protein, 10 aa, synthetic construct
  REGION 1..10: note = Synthetic Construct
GGGSGGSGGG

SEQ ID NO: 23 - protein, 13 aa, synthetic construct
  REGION 1..13: note = Synthetic Construct
GGGSGGSGGSGGG

SEQ ID NO: 24 - protein, 213 aa, synthetic construct
  REGION 1..213: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQQLYSALANKCCHVGCTKRSLARFCGGGSGGSGGGSWMEEVIKLCGRELVRAQIAICGMSTWS

SEQ ID NO: 25 - protein, 220 aa, synthetic construct
  REGION 1..220: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPGPAPGPAPGPAPGPAPGPAPGPAPGPAPGPAQLYSALANKCCHVGCTKRSLARFCGGGSGGSGGGSWMEEVIKLCGRELVRAQIAICGMSTWS

SEQ ID NO: 26 - protein, 213 aa, synthetic construct
  REGION 1..213: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQSWMEEVIKLCGRELVRAQIAICGMSTWSGGGSGGSGGGQLYSALANKCCHVGCTKRSLARFC

SEQ ID NO: 27 - protein, 220 aa, synthetic construct
  REGION 1..220: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPGPQPGPQPGPQPGPQPGPQPGPQPGPQPGPQSWMEEVIKLCGRELVRAQIAICGMSTWSGGGSGGSGGGQLYSALANKCCHVGCTKRSLARFC

SEQ ID NO: 28 - protein, 215 aa, synthetic construct
  REGION 1..215: note = Synthetic Construct
SWMEEVIKLCGRELVRAQIAICGMSTWSGGGSGGSGGGQLYSALANKCCHVGCTKRSLARFCGGGGQGGGGQGGGGQGGGGQGGGGQEVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPP

SEQ ID NO: 29 - protein, 222 aa, synthetic construct
  REGION 1..222: note = Synthetic Construct
SWMEEVIKLCGRELVRAQIAICGMSTWSGGGSGGSGGGQLYSALANKCCHVGCTKRSLARFCPGPQPGPQPGPQPGPQPGPQPGPQPGPQPGPQEVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPP

SEQ ID NO: 30 - protein, 213 aa, synthetic construct
  REGION 1..213: note = Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQSWMEEVIKLCGRELVRAQIAICGMSTWSGGGSGGSGGGQLYSALANKCCHVGCTKRSLARFC

SEQ ID NO: 31 - protein, 220 aa, synthetic construct
  REGION 1..220: note = Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSPGPQPGPQPGPQPGPQPGPQPGPQPGPQPGPQSWMEEVIKLCGRELVRAQIAICGMSTWSGGGSGGSGGGQLYSALANKCCHVGCTKRSLARFC

SEQ ID NO: 32 - protein, 215 aa, synthetic construct
  REGION 1..215: note = Synthetic Construct
SWMEEVIKLCGRELVRAQIAICGMSTWSGGGSGGSGGGQLYSALANKCCHVGCTKRSLARFCGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSPP

SEQ ID NO: 33 - protein, 222 aa, synthetic construct
  REGION 1..222: note = Synthetic Construct
SWMEEVIKLCGRELVRAQIAICGMSTWSGGGSGGSGGGQLYSALANKCCHVGCTKRSLARFCPGPQPGPQPGPQPGPQPGPQPGPQPGPQPGPQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSPP

SEQ ID NO: 34 - protein, 212 aa, synthetic construct
  REGION 1..212: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQSWMEEVIKLCGRELVRAQIAICGMSTWSGGGSGGSGGSGGGALANKCCHVGCTKRSLARFC

SEQ ID NO: 35 - protein, 212 aa, synthetic construct
  REGION 1..212: note = Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQSWMEEVIKLCGRELVRAQIAICGMSTWSGGGSGGSGGSGGGALANKCCHVGCTKRSLARFC

SEQ ID NO: 36 - protein, 209 aa, synthetic construct
  REGION 1..209: note = Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQALANKCCHVGCTKRSLARFCGGGSGGSGGGSWMEEVIKLCGRELVRAQIAICGMSTWS

SEQ ID NO: 37 - protein, 216 aa, synthetic construct
  REGION 1..216: note = Synthetic Construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCAARPGRPLITSKVADLYPYWGQGTLVTVSSPGPQPGPQPGPQPGPQPGPQPGPQPGPQPGPQALANKCCHVGCTKRSLARFCGGGSGGSGGGSWMEEVIKLCGRELVRAQIAICGMSTWS

SEQ ID NO: 38 - protein, 214 aa, synthetic construct
  REGION 1..214: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSGGGGQGGGGQGGGGQGGGGQGGGGQDSWMEEVIKLCGRELVRAQIAICGMSTWSSGGGGSGGGGQLYSALANKCCHVGCTKRSLARFC

SEQ ID NO: 39 - protein, 221 aa, synthetic construct
  REGION 1..221: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSSPGPAPGPAPGPAPGPAPGPAPGPAPGPAPGPADSWMEEVIKLCGRELVRAQIAICGMSTWSSGGGGSGGGGQLYSALANKCCHVGCTKRSLARFC

SEQ ID NO: 40 - protein, 757 aa, Homo sapiens
MTSGSVFFYILIFGKYFSHGGGQDVKCSLGYFPCGNITKCLPQLLHCNGVDDCGNQADEDNCGDNNGWSLQFDKYFASYYKMTSQYPFEAETPECLVGSVPVQCLCQGLELDCDETNLRAVPSVSSNVTAMSLQWNLIRKLPPDCFKNYHDLQKLYLQNNKITSISIYAFRGLNSLTKLYLSHNRITFLKPGVFEDLHRLEWLIIEDNHLSRISPPTFYGLNSLILLVLMNNVLTRLPDKPLCQHMPRLHWLDLEGNHIHNLRNLTFISCSNLTVLVMRKNKINHLNENTFAPLQKLDELDLGSNKIENLPPLIFKDLKELSQLNLSYNPIQKIQANQFDYLVKLKSLSLEGIEISNIQQRMFRPLMNLSHIYFKKFQYCGYAPHVRSCKPNTDGISSLENLLASIIQRVFVWVVSAVTCFGNIFVICMRPYIRSENKLYAMSIISLCCADCLMGIYLFVIGGFDLKFRGEYNKHAQLWMESTHCQLVGSLAILSTEVSVLLLTFLTLEKYICIVYPFRCVRPGKCRTITVLILIWITGFIVAFIPLSNKEFFKNYYGTNGVCFPLHSEDTESIGAQIYSVAIFLGINLAAFIIIVFSYGSMFYSVHQSAITATEIRNQVKKEMILAKRFFFIVFTDALCWIPIFVVKFLSLLQVEIPGTITSWVVIFILPINSALNPILYTLTTRPFKEMIHRFWYNYRQRKSMDSKGQKTYAPSFIWVEMWPLQEMPPELMKPDLFTYPCEMSLISQSTRLNSYS

SEQ ID NO: 41 - protein, 754 aa, Homo sapiens
MIVFLVFKHLFSLRLITMFFLLHFIVLINVKDFALTQGSMITPSCQKGYFPCGNLTKCLPRAFHCDGKDDCGNGADEENCGDTSGWATIFGTVHGNANSVALTQECFLKQYPQCCDCKETELECVNGDLKSVPMISNNVTLLSLKKNKIHSLPDKVFIKYTKLKKIFLQHNCIRHISRKAFFGLCNLQILYLNHNCITTLRPGIFKDLHQLTWLILDDNPITRISQRLFTGLNSLFFLSMVNNYLEALPKQMCAQMPQLNWVDLEGNRIKYLTNSTFLSCDSLTVLFLPRNQIGFVPEKTFSSLKNLGELDLSSNTITELSPHLFKDLKLLQKLNLSSNPLMYLHKNQFESLKQLQSLDLERIEIPNINTRMFQPMKNLSHIYFKNFRYCSYAPHVRICMPLTDGISSFEDLLANNILRIFVWVIAFITCFGNLFVIGMRSFIKAENTTHAMSIKILCCADCLMGVYLFFVGIFDIKYRGQYQKYALLWMESVQCRLMGFLAMLSTEVSVLLLTYLTLEKFLVIVFPFSNIRPGKRQTSVILICIWMAGFLIAVIPFWNKDYFGNFYGKNGVCFPLYYDQTEDIGSKGYSLGIFLGVNLLAFLIIVFSYITMFCSIQKTALQTTEVRNCFGREVAVANRFFFIVFSDAICWIPVFVVKILSLFRVEIPDTMTSWIVIFFLPVNSALNPILYTLTTNFFKDKLKQLLHKHQRKSIFKIKKKSLSTSIVWIEDSSSLKLGVLNKITLGDSIMKPVS

SEQ ID NO: 42 - protein, 469 aa, Homo sapiens
MQMADAATIATMNKAAGGDKLAELFSLVPDLLEAANTSGNASLQLPDLWWELGLELPDGAPPGHPPGSGGAESADTEARVRILISVVYWVVCALGLAGNLLVLYLMKSMQGWRKSSINLFVTNLALTDFQFVLTLPFWAVENALDFKWPFGKAMCKIVSMVTSMNMYASVFFLTAMSVTRYHSVASALKSHRTRGHGRGDCCGRSLGDSCCFSAKALCVWIWALAALASLPSAIFSTTVKVMGEELCLVRFPDKLLGRDRQFWLGLYHSQKVLLGFVLPLGIIILCYLLLVRFIADRRAAGTKGGAAVAGGRPTGASARRLSKVTKSVTIVVLSFFLCWLPNQALTTWSILIKFNAVPFSQEYFLCQVYAFPVSVCLAHSNSCLNPVLYCLVRREFRKALKSLLWRIASPSITSMRPFTATTKPEHEDQGLQAPAPPHAAAEPDLLYYPPGVVVYSGGRYDLLPSSSAY

SEQ ID NO: 43 - protein, 374 aa, Homo sapiens
MPTLNTSASPPTFFWANASGGSVLSADDAPMPVKFLALRLMVALAYGLVGAIGLLGNLAVLWVLSNCARRAPGPPSDTFVFNLALADLGLALTLPFWAAESALDFHWPFGGALCKMVLTATVLNVYASIFLITALSVARYWVVAMAAGPGTHLSLFWARIATLAVWAAAALVTVPTAVFGVEGEVCGVRLCLLRFPSRYWLGAYQLQRVVLAFMVPLGVITTSYLLLLAFLQRRQRRRQDSRVVARSVRILVASFFLCWFPNHVVTLWGVLVKFDLVPWNSTFYTIQTYVFPVTTCLAHSNSCLNPVLYCLLRREPRQALAGTFRDLRLRLWPQGGGWVQQVALKQVGRRWVASNPRESRPSTLLTNLDRGTPG

SEQ ID NO: 44 - protein, 20 aa, Mus musculus
METDTLLLWVLLLWVPGSTG

SEQ ID NO: 45 - protein, 126 aa, synthetic construct
  REGION 1..126: note = Synthetic Construct
EVQLLESGGGLVQPGGSLRLSCAASGRTVSSTAVAWFRQAPGKEREFVAGIGGSVDITYYADSVKGRFTISRDNSKNTLYLQMNSLRPEDTAVYYCAVRPGRPLITSRDANLYDYWGQGTLVTVSS

SEQ ID NO: 46 - protein, 126 aa, synthetic construct
  REGION 1..126: note = Synthetic Construct
EVQLLESGGGLVQPGGSLRLSCAASGRYIDSTAVAWFRQAPGKEREFVAGIGGGVDITYYADSVKGRFTISRDNSKNTLYLQMNSLRPEDTAVYYCAARPGRPLITSRVANLYPYWGQGTLVTVSS

SEQ ID NO: 47 - protein, 126 aa, synthetic construct
  REGION 1..126: note = Synthetic Construct
EVQLLESGGGLVQPGGSLRLSCAASYRYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSS

SEQ ID NO: 48 - protein, 126 aa, synthetic construct
  REGION 1..126: note = Synthetic Construct
EVQLLESGGGLVQPGGSLRLSCAASGAYIDETAVAWFRQAPGKEREFVAGIGGGVDITYYADSVKGRFTISRDNSKNTLYLQMNSLRPEDTAVYYCAARPGRPLITSKVADLYPYWGQGTLVTVSS

SEQ ID NO: 49 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDETYYADSVKGRFTISRDNSKNTLYLQMNSLRPEDTAVYYCAARPGRPLITSKVADLYPYWGQGTLVTVSS

SEQ ID NO: 50 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDQTYYADSVKGRFTISRDNSKNTLYLQMNSLRPEDTAVYYCAARPGRPLITSKVADLYPYWGQGTLVTVSS

SEQ ID NO: 51 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITAYADSVKGRFTISRDNSKNTLYLQMNSLRPEDTAVYYCAARPGRPLITSKVADLYPYWGQGTLVTVSS

SEQ ID NO: 52 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITEYADSVKGRFTISRDNSKNTLYLQMNSLRPEDTAVYYCAARPGRPLITSKVADLYPYWGQGTLVTVSS

SEQ ID NO: 53 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITQYADSVKGRFTISRDNSKNTLYLQMNSLRPEDTAVYYCAARPGRPLITSKVADLYPYWGQGTLVTVSS

SEQ ID NO: 54 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITSYADSVKGRFTISRDNSKNTLYLQMNSLRPEDTAVYYCAARPGRPLITSKVADLYPYWGQGTLVTVSS

SEQ ID NO: 55 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITTYADSVKGRFTISRDNSKNTLYLQMNSLRPEDTAVYYCAARPGRPLITSKVADLYPYWGQGTLVTVSS

SEQ ID NO: 56 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGKPLITSKVADLYPYWGQGTLVTVSS

SEQ ID NO: 57 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGQPLITSKVADLYPYWGQGTLVTVSS

SEQ ID NO: 58 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGSPLITSKVADLYPYWGQGTLVTVSS

SEQ ID NO: 59 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRELITSKVADLYPYWGQGTLVTVSS

SEQ ID NO: 60 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRQLITSKVADLYPYWGQGTLVTVSS

SEQ ID NO: 61 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRSLITSKVADLYPYWGQGTLVTVSS

SEQ ID NO: 62 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPEITSKVADLYPYWGQGTLVTVSS

SEQ ID NO: 63 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPGITSKVADLYPYWGQGTLVTVSS

SEQ ID NO: 64 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPQITSKVADLYPYWGQGTLVTVSS

SEQ ID NO: 65 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPTITSKVADLYPYWGQGTLVTVSS

SEQ ID NO: 66 - protein, 126 aa, synthetic construct
  REGION 1..126: note = Synthetic Construct
EVQLLESGGGLVQPGGSLRLSCAASGRYIDETAVAWFRQAPGKEREFVAGIGGGVDITYYADSVKGRFTISRDNSKNTLYLQMNSLRPEDTAVYYCAARPGRPLITEKVADLYPYWGQGTLVTVSS

SEQ ID NO: 67 - protein, 10 aa, synthetic construct
  REGION 1..10: note = Synthetic Construct
SGGGGSGGGG

(19) United States
(12) Patent Application Publication
(10) Pub. No.: US 2026/0125446 A1
(43) Pub. Date: May 7, 2026

(54) RELAXIN ANALOGS AND METHODS OF USING THE SAME

(71) Applicant: ELI LILLY AND COMPANY, Indianapolis, IN (US)

(72) Inventors: Petra VERDINO, San Diego, CA (US); Stacey Lynn LEE, San Diego, CA (US); Xiaojun WANG, Carmel, IN (US)

(21) Appl. No.: 19/243,335

(22) Filed: Jun. 19, 2025

Related U.S. Application Data
(63) Continuation of application No. 17/632,107, now Pat. No. 12,358,965.
(60) Provisional application No. 62/880,968, filed on Jul. 31, 2019.
(60) Provisional application No. 62/970,005, filed on Feb. 4, 2020.

(51) Int. Cl. C07K 14/64; A61K 31/155; A61K 31/426; A61K 31/64; A61K 38/22; A61K 38/30; A61K 39/00; A61K 45/06; A61K 47/68; A61P 3/10; C07K 16/18; C07K 16/28

(52) U.S. Cl. CPC C07K 14/64; A61K 31/155; A61K 31/426; A61K 31/64; A61K 38/22; A61K 38/30; A61K 45/06; A61K 47/6811; A61P 3/10; C07K 16/18; C07K 16/2863; A61K 2039/505; C07K 2317/92; C07K 2319/02; C07K 2319/31; C07K 2319/40

(57) ABSTRACT

Relaxin (RLN) analogs are disclosed including modifications that increase half-life when compared to native, human RLN, that maintain selectivity to the RXFP1 receptor and that provide in vitro and in vivo stability for improved druggability properties and less immunogenicity. Pharmaceutical compositions also are disclosed that include one or more of the RLN analogs described herein in a pharmaceutically acceptable carrier. Methods of making and using the RLN analogs also are disclosed, especially for treating cardiovascular, pulmonary and/or renal conditions, diseases or disorders.

The present application is being filed along with a Sequence Listing in ST.26 XML format. The Sequence Listing is provided as a file titled “X22483A_US_000 Sequence Listing ST26” created Jun. 11, 2025, and is 94,208 bytes in size. The Sequence Listing information in the ST.26 XML format is incorporated herein by reference in its entirety. No new matter is added herewith.

The disclosure relates generally to biology and medicine, and more particularly it relates to relaxin (RLN) analogs, especially long-acting, single-chain RLN analogs that can bind to a RLN/insulin-like family peptide (RXFP) receptor, such as the RXFP1 receptor, thereby functioning as RXFP receptor agonists. The disclosure further relates to compositions including the same and their use in treating cardiovascular, pulmonary and/or renal conditions, diseases or disorders.

Relaxins (RLNs) are part of the insulin superfamily and, in humans, include seven peptides of high structural, but low sequence, similarity—RLN1 (H1RLX, RLXH1 or H1), RLN2 (H2RLX, RLXH2 or H2), RLN3 (RXN3, ZINS4 or H3), insulin-like (INSL) peptide 3 (INSL3), INSL4, INSL5 and INSL6. Of particular interest herein is RLN2, which is a heterodimer of two peptide chains of twenty-four and twenty-nine amino acids (A chain and B chain), respectively, linked by two disulfide bonds with the A chain further having one intramolecular disulfide bond (see, Schwabe & McDonald (1977) Science 197:914-915). RLN2 is produced from its prohormone, prorelaxin, by cleaving a C peptide therefrom.

Physiologically, RLN2 exhibits a diverse array of functions that modulate cardiovascular, hepatic, neural, pancreatic, pulmonary and renal adaptations such as vasodilatory, anti-fibrotic and angiogenic effects, even though it initially was described as a pregnancy hormone. RLN2 signaling occurs through two different classes of G-protein-coupled receptors (GPCRs), namely, leucine-rich repeat-containing GPCRs LGR7 and LGR8, now referred to as the RXFP1 and RXFP2 receptors, respectively. Two other receptors in this family include the RXFP3 and RXFP4 receptors. RLN2 has a short half-life (t½), which presents challenges when using it as a therapeutic agent. In fact, native RLN2 has a t½ of minutes in vivo. Consequently, clinicians administer RLN2 by continuous intravenous infusion, which often results in inconvenience for individuals receiving the RLN compound and in short-term efficacy.

A number of RLN2 analogs exist having an improved t½. For example, Intl. Patent Application Publication No. WO 2018/148419 describes analogs that include a non-native amino acid residue such as para-acetyl-phenylalanine to which linkers, polymers and/or pharmacokinetic enhancers can be attached to improve t½. Intl. Patent Application Publication No. WO 2018/138170 describes analogs that are fusions of the A chain and B chain having a linker of at least fifteen amino acids and a half-life extending moiety to improve t½. Intl. Patent Application Publication No. WO 2017/201340 describes analogs that are fusions of the A chain and B chain having a variable light chain fragment to improve t2. Intl. Patent Application Publication No. WO 2015/067791 describes analogs that are carrier-linked prodrugs, especially PEG-based carriers, to improve t½ (see also, WO 2012/024452 for additional PEG-linked analogs). Intl. Patent Application Publication No. WO 2014/102179 describes analogs that are fusions of the A chain and B chain having a Fc moiety of IgG2 or IgG4 to improve t½. Intl. Patent Application Publication No. WO 2013/004607 describes analogs that are fusions of the A chain and B chain having a linker of at least five amino acids but less than fifteen amino acids to improve t½ or that are fusions of the A chain and B chain having a Fc domain of antibodies to improve t½.

In view of the increases in understanding the various physiological roles of RLNs, there remains a need for long-acting RLN analogs having an improved t½.

To address this need, the disclosure first describes single-chain RLN analogs having principal activity at the RXFP1 receptor (i.e., act as RXFP1 receptor agonists). Such RXFP1 receptor agonists include a basic structure from an amino-terminus (N-terminus) to a carboxy-terminus (C-terminus) of:

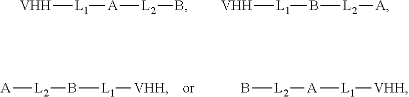

where VHH is a moiety that can act as a pharmacokinetic enhancer, A is a RLN A chain, B is a RLN B chain, L₁ is a first linker, and L₂ is a second linker.

In some instances, the VHH moiety can have an amino acid sequence of SEQ ID NO:10, 11, 12 or 13, especially SEQ ID NO:10 or 12. In other instances, the VHH moiety can have one or more additions, deletions, insertions or substitutions such that the VHH moiety has an amino acid sequence having at least about 90% to about 99% sequence similarity to any one of SEQ ID NOS:10, 11, 12 or 13 (see, e.g., SEQ ID NOS:45-66).

In some instances, the A chain can have an amino acid sequence of SEQ ID NO:2, 5 or 8, especially SEQ ID NO:5. In other instances, the A chain can have one or more additions, deletions, insertions or substitutions such that the A chain has an amino acid sequence having at least about 90% to about 99% sequence similarity to any one of SEQ ID NOS:2, 5 or 8. For example, the A chain can be des1-4 of SEQ ID NO:5.

In some instances, the B chain can have an amino acid sequence of SEQ ID NO:3, 6 or 9, especially SEQ ID NO:6. In other instances, the B chain can have one or more additions, deletions, insertions or substitutions such that the B chain has an amino acid sequence having at least about 90% to about 99% sequence similarity to any one of SEQ ID NOS:3, 6 or 9. For example, the B chain can be des1 of SEQ ID NO:6.

In some instances, L₁ can have an amino acid sequence of (GGGGQ)ₙ (SEQ ID NO:14), (GGGQ)ₙ (SEQ ID NO:15), (GGGGS)ₙ (SEQ ID NO:16), (PGPQ)ₙ (SEQ ID NO:17) or (PGPA)ₙ (SEQ ID NO:18), where n can be from 1 to 10, especially from about 5 to about 8. In other instances, L₁ can have an amino acid sequence of SEQ ID NO:19, 20 or 21. In still other instances, L₁ can have one or more additions, deletions, insertions or substitutions such that L₁ has an amino acid sequence having at least about 90% to about 99% sequence similarity to any one of SEQ ID NOS:14 to 21.

In some instances, L₂ can have an amino acid sequence of SEQ ID NO:22, 23 or 67. In other instances, L₂ can have one or more additions, deletions, insertions or substitutions.

In certain instances, the RLN analogs can have an amino acid sequence that includes a VHH of SEQ ID NO:10, 11, 12 or 13; an A chain of SEQ ID NO:2, 5 or 8; a B chain of SEQ ID NO:3, 6 or 9; a L₁ of SEQ ID NO:19, 20 or 21; and a L₂ of SEQ ID NO:22, 23 or 67. Alternatively, the RLN analogs can have an amino acid sequence having at least about 90% to about 99% sequence similarity to an amino acid sequence that includes an amino acid sequence that includes a VHH of SEQ ID NO:10, 11, 12 or 13; an A chain of SEQ ID NO:2, 5 or 8; a B chain of SEQ ID NO:3, 6 or 9; a L₁ of SEQ ID NO:19, 20 or 21; and a L₂ of SEQ ID NO:22, 23 or 67.

In particular instances, the RLN analogs can have an amino acid sequence of SEQ ID NO:24, 25, 26, 27, 28, 29, 30, 31, 32, 33, 34, 35, 36, 37, 38 or 39, especially SEQ ID NO:26, 27, 30, 31, 34 or 35. Alternatively, the RLN analogs can have an amino acid sequence having at least about 90% to about 99% sequence similarity to an amino acid sequence of SEQ ID NO:24, 25, 26, 27, 28, 29, 30, 31, 32, 33, 34, 35, 36, 37, 38 or 39, especially SEQ ID NO:26, 27, 30, 31, 34 or 35.

In some instances, the RLN analogs have a binding affinity at a RXFP1 receptor that is comparable to the binding affinity of native, human RLN2 (SEQ ID NOS:5 and 6). In other instances, the RLN analogs have a binding affinity at a RXFP1 receptor that is greater than that of native, human RLN2 (SEQ ID NOS:5 and 6). In still other instances, the RLN analogs have a binding affinity at a RXFP1 receptor that is less than that of native, human RLN2 (SEQ ID NOS:5 and 6).

In some instances, the RLN analogs have a t½ that is longer than that of native, human RLN2 (SEQ ID NOS:5 and 6), including up to about 20 days to about 30 days longer when administered to a human.

The compositions above alternatively can be nucleic acid sequences encoding the amino acid sequences described herein, as well as vectors and host cells including the same for expressing the RLN analogs herein.

Second, pharmaceutical compositions are described that include at least one RLN analog herein or a pharmaceutically acceptable salt thereof (e.g., trifluroacetate salts, acetate salts or hydrochloride salts) and a pharmaceutically acceptable carrier. In some instances, the pharmaceutically acceptable carrier is a buffer such as, for example, physiological saline, phosphate-buffered saline, citrate-buffered saline or histidine-buffered saline. In certain instances, the buffer is histidine, a histidine buffer or a histidine-buffered saline. In other instances, the pharmaceutical compositions further can include carriers, diluents and/or excipients.

Moreover, the pharmaceutical compositions can include at least one additional therapeutic agent such as, for example, an agent used as a standard of care in a cardiovascular, pulmonary and/or renal condition, disease or disorder. In some instances, the at least one additional therapeutic agent can be an anticoagulant, an angiotensin-converting enzyme (ACE) inhibitor, an angiotensin II receptor blocker (ARB), an ARB neprilysin inhibitor (ARNI), a β-blocker, a diuretic, digitalis, digoxin, hydralazine/isorbide dinitrate, a mineralocorticoid receptor antagonist (MRA; or aldosterone antagonist), a sodium-glucose cotransporter-2 (SGLT2) inhibitor, a statin and/or an anti-glycemic agent.

Third, methods are described for using the RLN analogs herein, especially for using the RLN analogs to treat cardiovascular, pulmonary and/or renal conditions, diseases or disorders. The methods include at least a step of administering to an individual in need thereof an effective amount of at least one RLN analog herein or a pharmaceutically acceptable salt thereof.

In some instances, the RLN analog can be administered via any standard route of administration such as, for example, intramuscularly, intravenously, parenterally, subcutaneously or transdermally. In certain instances, the RLN analog is administered subcutaneously (SQ), intramuscularly (IM) or intravenously (IV). In particular instances, the RLN analog can be administered SQ or IV to the individual.

Likewise, and in some instances, the RLN analog can be administered daily, every other day, three times a week, two times a week, one time a week (i.e., weekly), biweekly (i.e., every other week), one time a month (i.e., monthly), bimonthly (i.e., every other month), or even every three months. In certain instances, the RLN analog can be administered SQ every other day, SQ three times a week, SQ two times a week, SQ one time a week, SQ every other week, or SQ once a month. In particular instances, the RLN analog is administered SQ one time a week (QW).

Alternatively, the RLN analog can be IV administered to the individual. As above, the RLN analog can be administered daily, every other day, three times a week, two times a week, one time a week (i.e., weekly), biweekly (i.e., every other week), or monthly. In certain instances, the RLN analog can be administered IV every other day, IV three times a week, IV two times a week, IV one time a week, IV every other week, or IV once a month. In particular instances, the RLN analog is administered IV one time a week.

The methods also can include a step of administering the RLN analog in combination with an effective amount of at least one additional therapeutic agent. Briefly, the standard of care for many of the conditions/diseases/disorders herein includes an anticoagulant, an ACE inhibitor, an ARB, an ARNI, a β-blocker, a diuretic, digitalis, digoxin, hydralazine/isorbide dinitrate, a MRA or other aldosterone antagonist, a SGLT2 inhibitor, a statin and/or an anti-glycemic agent, as well as other therapeutic agents to control comorbidities, including, but not limited to, high cholesterol, high blood pressure, atrial fibrillation and diabetes. In some instances, the additional therapeutic agent can be administered simultaneously, separately or sequentially with the RLN analog.

For example, the additional therapeutic agent can be administered with a frequency the same as the RLN analog (i.e., every other day, twice a week, weekly or even monthly). In other instances, the additional therapeutic agent can be administered with a frequency distinct from the RLN analog. In other instances, the additional therapeutic agent can be administered SQ or IV. In still other instances, the RLN analog is administered SQ, and the additional therapeutic agent can be administered orally or IV. Alternatively, the RLN analog is administered IV, and the additional therapeutic agent is administered SQ.

In some instances, the individual in need is a diabetic, hypertensive with kidney function impairment and/or obese.

The methods also may include steps such as measuring or obtaining blood pressure and comparing such obtained values to one or more baseline values or previously obtained values to assess the effectiveness of treatment/therapy.

The methods also may be combined with diet and exercise and/or may be combined with additional therapeutic agents other than those discussed above.

Fourth, uses are described that include at least one of the RLN analogs herein. For example, the RLN analogs herein can be provided for use in therapy, especially in treating cardiovascular, pulmonary and/or renal conditions, diseases or disorders. The RLN analogs optionally can be administered simultaneously, separately or sequentially (i.e., in combination) with at least one additional therapeutic agent. Likewise, use of the RLN analogs herein is provided in manufacturing a medicament for treating cardiovascular, pulmonary and/or renal conditions, diseases or disorders, where the medicament optionally may further include one or more additional therapeutic agents as noted above.

Fifth, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSS (SEQ ID NO:10). In some instances, the compound can have an amino acid sequence having at least about 90% to about 99% sequence similarity to SEQ ID NO:10.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSSPP (SEQ ID NO:11). In some instances, the compound can have an amino acid sequence having at least about 90% to about 99% sequence similarity to SEQ ID NO:11.

Alternatively, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV ADLYPYWGQGTLVTVSS (SEQ ID NO:12). In some instances, the compound can have an amino acid sequence having at least about 90% to about 99% sequence similarity to SEQ ID NO:12.

Alternatively, a compound is provided that includes an amino acid sequence of: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV ADLYPYWGQGTLVTVSSPP (SEQ ID NO: 13). In some instances, the compound can have an amino acid sequence having at least about 90% to about 99% sequence similarity to SEQ ID NO:13.

Alternatively, a compound is provided that includes an amino acid sequence of: EVQLLESGGGLVQPGGSLRLSCAASGRTVSSTAVAWFRQAPGKEREFVAGIGGS VDITYYADSVKGRFTISRDNSKNTLYLQMNSLRPEDTAVYYCAVRPGRPLITSRD ANLYDYWGQGTLVTVSS (SEQ ID NO:45). In some instances, the compound can have an amino acid sequence having at least about 90% to about 99% sequence similarity to SEQ ID NO:45.

Alternatively, a compound is provided that includes an amino acid sequence of: EVQLLESGGGLVQPGGSLRLSCAASGRYIDSTAVAWFRQAPGKEREFVAGIGGG VDITYYADSVKGRFTISRDNSKNTLYLQMNSLRPEDTAVYYCAARPGRPLITSRV ANLYPYWGQGTLVTVSS (SEQ ID NO:46). In some instances, the compound can have an amino acid sequence having at least about 90% to about 99% sequence similarity to SEQ ID NO:46.

Alternatively, a compound is provided that includes an amino acid sequence of: EVQLLESGGGLVQPGGSLRLSCAASYRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSS (SEQ ID NO:47). In some instances, the compound can have an amino acid sequence having at least about 90% to about 99% sequence similarity to SEQ ID NO:47.

Alternatively, a compound is provided that includes an amino acid sequence of: EVQLLESGGGLVQPGGSLRLSCAASGAYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSS (SEQ ID NO:48). In some instances, the compound can have an amino acid sequence having at least about 90% to about 99% sequence similarity to SEQ ID NO:48.

Alternatively, a compound is provided that includes an amino acid sequence of: EVQLLESGGGLVQPGGSLRLSCAASGRYIDETAVAWFRQAPGKEREFVAGIGGG VDETYYADSVKGRFTISRDNSKNTLYLQMNSLRPEDTAVYYCAARPGRPLITSKV ADLYPYWGQGTLVTVSS (SEQ ID NO:49). In some instances, the compound can have an amino acid sequence having at least about 90% to about 99% sequence similarity to SEQ ID NO:49.

Alternatively, a compound is provided that includes an amino acid sequence of: EVQLLESGGGLVQPGGSLRLSCAASGRYIDETAVAWFRQAPGKEREFVAGIGGG VDQTYYADSVKGRFTISRDNSKNTLYLQMNSLRPEDTAVYYCAARPGRPLITSK VADLYPYWGQGTLVTVSS (SEQ ID NO:50). In some instances, the compound can have an amino acid sequence having at least about 90% to about 99% sequence similarity to SEQ ID NO:50.

Alternatively, a compound is provided that includes an amino acid sequence of: EVQLLESGGGLVQPGGSLRLSCAASGRYIDETAVAWFRQAPGKEREFVAGIGGG VDITAYADSVKGRFTISRDNSKNTLYLQMNSLRPEDTAVYYCAARPGRPLITSKV ADLYPYWGQGTLVTVSS (SEQ ID NO:51). In some instances, the compound can have an amino acid sequence having at least about 90% to about 99% sequence similarity to SEQ ID NO:51.

Alternatively, a compound is provided that includes an amino acid sequence of: EVQLLESGGGLVQPGGSLRLSCAASGRYIDETAVAWFRQAPGKEREFVAGIGGG VDITEYADSVKGRFTISRDNSKNTLYLQMNSLRPEDTAVYYCAARPGRPLITSKV ADLYPYWGQGTLVTVSS (SEQ ID NO:52). In some instances, the compound can have an amino acid sequence having at least about 90% to about 99% sequence similarity to SEQ ID NO:52.

Alternatively, a compound is provided that includes an amino acid sequence of: EVQLLESGGGLVQPGGSLRLSCAASGRYIDETAVAWFRQAPGKEREFVAGIGGG VDITQYADSVKGRFTISRDNSKNTLYLQMNSLRPEDTAVYYCAARPGRPLITSKV ADLYPYWGQGTLVTVSS (SEQ ID NO:53). In some instances, the compound can have an amino acid sequence having at least about 90% to about 99% sequence similarity to SEQ ID NO:53.

Alternatively, a compound is provided that includes an amino acid sequence of: EVQLLESGGGLVQPGGSLRLSCAASGRYIDETAVAWFRQAPGKEREFVAGIGGG VDITSYADSVKGRFTISRDNSKNTLYLQMNSLRPEDTAVYYCAARPGRPLITSKV ADLYPYWGQGTLVTVSS (SEQ ID NO:54). In some instances, the compound can have an amino acid sequence having at least about 90% to about 99% sequence similarity to SEQ ID NO:54.

Alternatively, a compound is provided that includes an amino acid sequence of: EVQLLESGGGLVQPGGSLRLSCAASGRYIDETAVAWFRQAPGKEREFVAGIGGG VDITTYADSVKGRFTISRDNSKNTLYLQMNSLRPEDTAVYYCAARPGRPLITSKV ADLYPYWGQGTLVTVSS (SEQ ID NO:55). In some instances, the compound can have an amino acid sequence having at least about 90% to about 99% sequence similarity to SEQ ID NO:55.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGKPLITSKV ADLYPYWGQGTLVTVSS (SEQ ID NO:56). In some instances, the compound can have an amino acid sequence having at least about 90% to about 99% sequence similarity to SEQ ID NO:56.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGQPLITSKV ADLYPYWGQGTLVTVSS (SEQ ID NO:57). In some instances, the compound can have an amino acid sequence having at least about 90% to about 99% sequence similarity to SEQ ID NO:57.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGSPLITSKV ADLYPYWGQGTLVTVSS (SEQ ID NO:58). In some instances, the compound can have an amino acid sequence having at least about 90% to about 99% sequence similarity to SEQ ID NO:58.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRELITSKV ADLYPYWGQGTLVTVSS (SEQ ID NO:59). In some instances, the compound can have an amino acid sequence having at least about 90% to about 99% sequence similarity to SEQ ID NO:59.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRQLITSKV ADLYPYWGQGTLVTVSS (SEQ ID NO:60). In some instances, the compound can have an amino acid sequence having at least about 90% to about 99% sequence similarity to SEQ ID NO:60.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRSLITSKV ADLYPYWGQGTLVTVSS (SEQ ID NO:61). In some instances, the compound can have an amino acid sequence having at least about 90% to about 99% sequence similarity to SEQ ID NO:61.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPEITSKV ADLYPYWGQGTLVTVSS (SEQ ID NO:62). In some instances, the compound can have an amino acid sequence having at least about 90% to about 99% sequence similarity to SEQ ID NO:62.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPGITSKV ADLYPYWGQGTLVTVSS (SEQ ID NO:63). In some instances, the compound can have an amino acid sequence having at least about 90% to about 99% sequence similarity to SEQ ID NO:63.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPQITSKV ADLYPYWGQGTLVTVSS (SEQ ID NO:64). In some instances, the compound can have an amino acid sequence having at least about 90% to about 99% sequence similarity to SEQ ID NO:64.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPTITSKV ADLYPYWGQGTLVTVSS (SEQ ID NO:65). In some instances, the compound can have an amino acid sequence having at least about 90% to about 99% sequence similarity to SEQ ID NO:65.

Alternatively, a compound is provided that includes an amino acid sequence of: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITEKV ADLYPYWGQGTLVTVSS (SEQ ID NO:66). In some instances, the compound can have an amino acid sequence having at least about 90% to about 99% sequence similarity to SEQ ID NO:66.

An advantage of the RLN analogs herein is that they can be chemically or recombinantly synthesized as a single-chain polypeptide (i.e., monomeric) and thus do not require endoproteolytic processing for biological activity. It is contemplated, however, that in some instances, the VHH moiety can be conjugated not only to single-chain RLNs but also to two-chain RLNs (e.g., native). On the VHH moiety, one could conjugate not only to the N- and C-terminus but also to any surface-exposed amino acid of the VHH (with the proviso that such conjugation does not entirely abrogate albumin binding).

An advantage of the RLN analogs herein is that the VHH moieties can be used not only with native A chain and B chain sequences but also with modified sequences thereof. Moreover, the VHH moieties may be further modified to have enhanced or additional functionality via other peptide/protein fusions or small molecules being attached to the VHH moieties.

An advantage of the RLN analogs herein is that the VHH moieties provide an extended duration of action in mammals such as humans and can have a t½ of about 20 days to about 30 days, thereby allowing for at least weekly or biweekly administration when compared to native, human RLN, especially native, human RLN2 (SEQ ID NOS:5 and 6), which can improve compliance.

An advantage of the RLN analogs herein is that they have similar or better selectivity, affinity and/or potency for RXFP1 than RXFP2 receptors when compared to native, human RLN2 (SEQ ID NOS:5 and 6). Alternatively stated, the RLN analogs herein result in sufficient activity at RXFP1 receptor and reduced or insufficient activity at one or more of the RXFP2, RXFP3 and RXFP4 receptors.

An advantage of the RLN analogs herein is that they have tunable pharmacokinetics achieved by changing albumin affinity of the VHH moieties.

An advantage of the RLN analogs herein is that they have improved stability in a preserved formulation when compared to native, human RLN2 (SEQ ID NOS:5 and 6) or RLN analogs having an Fc fusion.

Moreover, an advantage of the VHH moieties is that they have equal binding not only to human serum albumin but also to dog, monkey, mouse, pig and rat serum albumin, which allows for pharmacodynamic, pharmacokinetic and toxicology studies to more readily translate from these species to humans.

An advantage of the VHH moieties is that they not only can be used to improve the t½ of the RLN analogs herein when compared to native, human RLN2 (SEQ ID NOS:5 and 6) but also can be used to improve the t½ of other biologically active peptides and proteins such as, for example, insulin, growth differentiation factor 15 (GDF-15) or glucose-dependent insulinotropic peptide 1 (GLP-1).

Unless defined otherwise, all technical and scientific terms used herein have the same meaning as commonly understood by one of skill in the art to which the disclosure pertains. Although any methods and materials similar to or equivalent to those described herein can be used in the practice or testing of the RLN analogs, pharmaceutical compositions and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

### Definitions

As used herein, “about” means within a statistically meaningful range of a value or values such as, for example, a stated concentration, length, molecular weight, pH, sequence similarity, time frame, temperature, volume, etc. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, and in reference to one or more of the RXFP receptors, “activity,” “activate,” “activating” and the like means a capacity of a compound, such as a RLN analog herein, to bind to and induce a response at its receptor(s), as measured using assays known in the art, such as the in vitro assays described below.

As used herein, “amino acid” means a molecule that, from a chemical standpoint, is characterized by a presence of one or more amine groups and one or more carboxylic acid groups, and may contain other functional groups. As is known in the art, there is a set of twenty amino acids that are designated as standard amino acids and that can be used as building blocks for most of the peptides/proteins produced by any living being. The amino acid sequences in the disclosure contain the standard single letter or three letter codes for the twenty standard amino acids.

As used herein, “analog” means a compound, such as a synthetic peptide or polypeptide, that activates a target receptor and that elicits at least one in vivo or in vitro effect elicited by a native agonist for that receptor.

As used herein, “conservative substitution” means a variant of a reference peptide or polypeptide that is identical to the reference molecule, except for having one or more conservative amino acid substitutions in its amino acid sequence. In general, a conservatively modified variant includes an amino acid sequence that is at least about 70%, 75%, 80%, 85%, 90%, 91%, 92%, 93%, 94%, 95%, 96%, 97%, 98% or 99% identical to a reference amino acid sequence. More specifically, a conservative substitution refers to substitution of an amino acid with an amino acid having similar characteristics (e.g., charge, side-chain size, hydrophobicity/hydrophilicity, backbone conformation and rigidity, etc.) and having minimal impact on the biological activity of the resulting substituted peptide or polypeptide. Conservative substitutions of functionally similar amino acids are well known in the art and thus need not be exhaustively described herein.

As used herein, “effective amount” means an amount or dose of one or more RLN analogs herein, or a pharmaceutically acceptable salt thereof that, upon single or multiple dose administration to an individual in need thereof, provides a desired effect in such an individual under diagnosis or treatment (i.e., may produce a clinically measurable difference in a condition of the individual such as, for example, increased angiogenesis, increased vascular compliance, increased cardiac blood flow, increased hepatic blood flow, increased pulmonary blood flow, increased renal blood flow, increased glomerular filtration rate, decreased blood pressure, decreased (or prevented) inflammation and/or reduced (or prevented) fibrosis in the heart, kidney, liver or lung). An effective amount can be readily determined by one of skill in the art by using known techniques and by observing results obtained under analogous circumstances. In determining the effective amount for an individual, a number of factors are considered, including, but not limited to, the species of mammal, its size, age and general health, the specific disease or disorder involved, the degree of or involvement or the severity of the disease or disorder, the response of the individual, the particular RLN analog administered, the mode of administration, the bioavailability characteristics of the preparation administered, the dose regimen selected, the use of concomitant medication, and other relevant circumstances.

As used herein, “extended duration of action” means that binding affinity and activity for a RLN analog herein continues for a period of time greater than a native RLN, especially native, human RLN2 (SEQ ID NOS:5 and 6), allowing for dosing at least as infrequently as once daily or even thrice-weekly, twice-weekly or once-weekly. The time action profile of the RLN analog may be measured using known pharmacokinetic test methods such as those utilized in the Examples below.

As used herein, “half-life” or “t½” means a time it takes for one-half of a quantity of a compound, such as native RLN or a RLN analog herein, to be removed from a fluid or other physiological space such as serum or plasma of an individual by biological processes. Alternatively, t½ also can mean a time it takes for a quantity of such a compound to lose one-half of its pharmacological, physiological or radiological activity.

As used herein, “half-maximal effective concentration” or “EC₅₀” means a concentration of compound that results in 50% activation/stimulation of an assay endpoint, such as a dose-response curve (e.g., cAMP, PI3K-Akt, NFκβ, VEGF and/or nitric oxide (NO) signaling pathways).

As used herein, “in combination with” means administering at least one of the RLN analogs herein either simultaneously, sequentially or in a single combined formulation with one or more additional therapeutic agents.

As used herein, “individual in need thereof” means a mammal, such as a human, with a condition, disease, disorder or symptom requiring treatment or therapy, including for example, those listed herein. In particular, the individual to be treated is a human.

As used herein, “long-acting” means that binding affinity and activity of a RLN analog herein continues for a period of time greater than native, human RLN2 (SEQ ID NOS:5 and 6), allowing for dosing at least as infrequently as once daily or even thrice-weekly, twice-weekly, once-weekly or monthly. The time action profile of the RLN analogs may be measured using known pharmacokinetic test methods such as those described in the Examples below.

As used herein, “non-standard amino acid” means an amino acid that may occur naturally in cells but does not participate in peptide synthesis. Non-standard amino acids can be constituents of a peptide and often are generated by modifying standard amino acids in the peptide (i.e., via post-translational modification). Non-standard amino acids can include D-amino acids, which have an opposite absolute chirality of the standard amino acids above.

As used herein, “pharmaceutically acceptable buffer” means any of the standard pharmaceutical buffers known to one of skill in the art.

As used herein, “RLN” means a relaxin obtained or derived from any species, such as a mammalian species, especially a human, where the native form is a heterodimeric peptide having two peptide chains (e.g., an A chain and a B chain) connected via two disulfide bonds, and with the A chain further having a single intramolecular disulfide bond. RLN includes both native RLN (i.e., full-length) and variations thereof (i.e., additions, deletions, insertions and/or substitutions of native RLN). In humans, there are three native RLN isoforms—RLN1, RLN2 and RLN3. RLN processing begins with preprorelaxin, which is processed to prorelaxin (includes A chain, B chain and C peptide; native RLN has a structure of B-C-A), where the sequence of native, human proRLN1 is set forth in SEQ ID NO:1 (see also, UniProt/SwissProt Database Accession No. P04808), the sequence of native, human proRLN2 is set forth in SEQ ID NO:4 (see also, UniProt/SwissProt Database Accession No. P04090), and the sequence of native, human proRLN3 is set forth in SEQ ID NO:7 (see also, UniProt/SwissProt Database Accession No. Q8WXF3). Prorelaxin undergoes further processing in which the C peptide is cleaved to arrive at RLN. The sequences for the A chain of native, human RLN1, RLN2 and RLN3 are set forth in SEQ ID NOS:2, 5 and 8, respectively. Likewise, the sequences for the B chain of native, human RLN1, RLN2 and RLN3 are set forth in SEQ ID NOS:3, 6 and 9, respectively.

In humans, there are four RLN receptors—RXFP1 (SEQ ID NO:40; see also, UniProt/SwissProt Database Accession No. Q9HBX9), RXFP2 (SEQ ID NO:41; see also, UniProt/SwissProt Database Accession No. Q8WXD0), RXFP3 (SEQ ID NO:42; see also, UniProt/SwissProt Database Accession No. Q9NSD7) and RXFP4 (SEQ ID NO:43; see also, UniProt/SwissProt Database Accession No. Q8TDU9)—that act as GPCRs (see, Halls et al. (2007) Br. J. Pharmacol. 150:677-691). Of interest herein are the RXFP1 and RXFP2 receptors, both of which can bind RLN1 and RLN2. The RXFP1 receptor has been found in the brain, blood cells, bone, heart, kidney, lung, liver and vasculature, whereas the RXFP2 receptor is much more restricted and has been found in the bone and gubemaculum. Stimulation of the RXFP1 and RXFP2 receptors activates signal transduction networks involving adenylate cyclase, protein kinase A, protein kinase C, phosphatidylinositol 3-kinase and/or extracellular signal-regulated kinases (Erk1/2).

As used herein, “RLN analog” and the like means a compound, such as a peptide or polypeptide, that elicits one or more effects of native RLN at one or more RXFP receptors but varies in some manner with respect to the amino acid sequence when compared native RLN. RLN analog also can include variants of these compounds, which are functionally equivalent to RLN but have sequences that are fragments or are the complete sequence but having additions, deletions, insertions and/or substitutions. All references to amino acid positions in unmodified or modified RLNs described herein are based on the corresponding position in the A chain of SEQ ID NO:5 or the B chain of SEQ ID NO:6 of native, human RLN2, unless otherwise specified. In some instances, the RLN analogs herein can bind to a RXFP with higher or lower affinity but demonstrate a longer t½ in vivo or in vitro when compared to native RLN, especially a native, human RLN2 (SEQ ID NOS:5 and 6). In this manner, the RLN analogs herein are synthetic compounds that act as RXFP receptor agonists.

As used herein, “sequence similarity” means a quantitative property of two or more nucleic acid sequences or amino acid sequences of biological compounds such as, for example, a correspondence over an entire length or a comparison window of the two or more sequences. Sequence similarity can be measured by (1) percent identity or (2) percent similarity. Percent identity measures a percentage of residues identical between two biological compounds divided by the length of the shortest sequence; whereas percent similarity measures identities and, in addition, includes sequence gaps and residue similarity in the evaluation. Methods of and algorithms for determining sequence similarity are well known in the art and thus need not be exhaustively described herein. A specified percentage of identical nucleotide or amino acid positions is at least about 75%, 80%, 85%, 86%, 87%, 88%, 89%, 90%, 91%, 92%, 93%, 94%, 95%, 96%, 97%, 98%, 99% or higher.

As used herein, “single-chain relaxin,” “scRLN” and the like means a RLN polypeptide where the A and B chains are connected to one another by a linker (i.e., L₂) as in A-L₂-B or B-L₂-A. Moreover, scRLN can include at least one of the native interchain and/or intrachain disulfide bonds to maintain correct structural folding.

As used herein, “two-chain relaxin,” “tcRLN” and the like means a RLN polypeptide where the A and B chains are connected to one another by one or more interchain and/or intrachain disulfide bonds, but not by any linkers, to maintain correct structural folding, such as a native RLN.

As used herein, “treating” or “to treat” means managing and caring for an individual having a condition, disease, disorder or symptom for which RLN analog administration is indicated for the purpose of attenuating, restraining, reversing, slowing or stopping progression or severity of the condition, disease, disorder and/or symptom. Treating includes administering a RLN analog herein or composition containing a RLN analog herein to the individual to prevent the onset of symptoms or complications, alleviating the symptoms or complications, or eliminating the condition, disease, disorder or symptom. Treating includes administering a RLN analog or composition containing a RLN analog herein to the individual to result in such as, for example, increased angiogenesis, increased vascular compliance, increased cardiac blood flow, increased hepatic blood flow, increased pulmonary blood flow, increased renal blood flow, increased glomerular filtration rate, decreased blood pressure, decreased (or prevented) inflammation and/or reduced (or prevented) fibrosis in the heart, kidney, liver or lung). The individual to be treated is a mammal, especially a human.

As used herein, “individual,” “patient” and “subject” are used interchangeably and mean a mammal, especially a human. In certain instances, the individual is further characterized with a condition, disease, disorder and/or symptom that would benefit from administering a RLN analog herein.

As used herein, “VHH” or “VHH moiety” means a form of single-domain antibody, especially an antibody fragment of a single, monomeric variable region of a heavy chain only antibody (HcAb), which has a very small size of about 15 kDa. It has been found herein that VHH moieties can be used as a pharmacokinetic enhancer to extend the duration of action of and/or to improve the t½ of the RLN analogs herein. The VHH moieties herein bind serum albumin; however, the VHH moieties can be used to bind IgG (including Fc domain), neonatal Fc receptor (FcRn) or other long-lasting serum proteins. Although the VHH moieties herein are used to improve the t½ of RLN, they likewise can be used to improve the t½ of other biologically active peptides/proteins such as, for example, insulin, GDF-15 or GLP-1.

Certain abbreviations are defined as follows: “ACR” refers to urine albumin/urine creatinine ratio; “amu” refers to atomic mass unit; “AUC” refers to area under the curve; “Boc” refers to tert-butoxycarbonyl; “cAMP” refers to cyclic adenosine monophosphate; “CMV” refers to cytomegalovirus; “CV” refers to column volume; “DNA” refers to deoxyribonucleic acid; “DMF” refers to dimethylformamide; “DMSO” refers to dimethyl sulfoxide; “EDC” refers to 1-ethyl-3-(3-dimethylaminopropyl) carbodiimide hydrochloride; “EDTA” refers to ethylenediaminetetraacetic acid; “EIA/RIA” refers to enzyme immunoassay/radioimmunoassay; “ETA” refers to ethanolamine; “GS” refers to glutamine synthetase; “HIC” refers to hydrophobic interaction chromatography; “hr” refers to hour or hours; “HTRF” refers to homogenous time-resolved fluorescent; “IV” refers to intravenous; “IP” refers to intraperitoneal; “kDa” refers to kilodaltons; “LC/MS” refers to liquid chromatography-mass spectrometry; “min” refers to minute or minutes; “MS” refers to mass spectrometry; “MSX” refers to methionine sulfoximine; “NaOAc” refers to sodium acetate; “NHS” refers to N-hydroxysuccinimide; “OtBu” refers to O-tert-butyl; “Pbf” refers to NG-2,2,4,6,7-pentamethyldihydrobenzofuran-5-sulfonyl; “PEI” refers to polyethylenimine; “RP-HPLC” refers to reversed-phase high performance liquid chromatography; “RU” means resonance units; “sec” refers to second or seconds; “SPR” refers to surface plasmon resonance; “SQ” refers to subcutaneous; “SEC” refers to size-exclusion chromatography; “SEM” refers to standard error of the mean; “TFA” refers to trifluoroacetic acid; and “Trt” refers to trityl.

### RLN Analogs

The RLN analogs herein have structural similarities to, but many structural differences, from native, human RLN. For example, when compared to native, human RLN2 (SEQ ID NOS:5 and 6), the RLN analogs lack one or more of the amino acids present in native, human RLN2, include a peptide linker between the A chain and the B chain, and include an albumin-binding VHH moiety. The RLN analogs result in sufficient activity at the RXFP1 receptor and reduced or insufficient activity at one or more of the RXFP2, RXFP3 and RXFP4 receptors. Likewise, the RLN analogs have beneficial attributes relevant to their developability as therapeutic treatments, including improved solubility in aqueous solutions, improved chemical and physical formulation stability, extended pharmacokinetic profile (which can be tuned based upon VHH affinity to serum albumin), and/or minimized potential for immunogenicity.

Briefly, the RLN analogs herein include an amino acid sequence from the N-terminus to the C-terminus having one of the following structures:

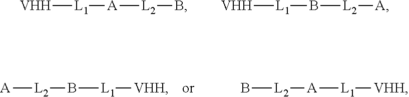

where VHH is a moiety acting as a pharmacokinetic enhancer, A is a RLN A chain, B is a RLN B chain, L₁ is a first peptide linker and L₂ is a second peptide linker, where L₁ and L₂ are distinct from one another (i.e., each have an amino acid sequence that is not the same).

With regard to the A chain, it can be a native RLN A chain, such as a native, human RLN1 A chain (SEQ ID NO:2); native, human RLN2 A chain (SEQ ID NO:5); or native, human RLN3 A chain (SEQ ID NO:8). Alternatively, the A chain can be a variant thereof. For example, one A chain variant can have an amino acid sequence that lacks residues 1 to 4 of SEQ ID NO:5 (i.e., des1-4 human RLN2 A chain or desA1-4).

Likewise, and with regard to the B chain, it can be a native RLN B chain, such as a native, human RLN1 B chain (SEQ ID NO:3); native, human RLN2 B chain (SEQ ID NO:6); or native, human RLN3 B chain (SEQ ID NO: 9). Alternatively, the B chain can be a variant thereof. For example, one B chain variant can have an amino acid sequence that lacks residue 1 of SEQ ID NO:6 (i.e., des1 human RLN2 B chain or desB1).

In some instances, the A chain can be a native, human RLN1 A chain (SEQ ID NO:2) and the B chain can be a native, human RLN1 B chain (SEQ ID NO:3); the A chain can be a native, human RLN2 A chain (SEQ ID NO:5) and the B chain can be a native, human RLN2 B chain (SEQ ID NO:6); the A chain can be a native, human RLN3 A chain (SEQ ID NO:8) and the B chain can be a native, human RLN3 B chain (SEQ ID NO:9); the A chain can be a native, human RLN1 A chain (SEQ ID NO:2) and the B chain can be a native, human RLN2 B chain (SEQ ID NO:6); the A chain can be a native, human RLN1 A chain (SEQ ID NO:2) and the B chain can be a native, human RLN3 B chain (SEQ ID NO:9); the A chain can be a native, human RLN2 A chain (SEQ ID NO:5) and the B chain can be a native, human RLN1 B chain (SEQ ID NO:3); the A chain can be a native, human RLN2 A chain (SEQ ID NO:5) and the B chain can be a native, human RLN3 B chain (SEQ ID NO:9); the A chain can be a native, human RLN3 A chain (SEQ ID NO:8) and the B chain can be a native, human RLN1 B chain (SEQ ID NO:3); or the A chain can be a native, human RLN3 A chain (SEQ ID NO:8) and the B chain can be a native, human RLN2 B chain (SEQ ID NO:6).

In some instances, the A chain may be a RLN2 A chain variant that lacks residues 1 to 4 (desA1-4) and the B chain may be any native B chain. In other instances, the A chain may be any native A chain and the B chain may be a RLN2 B chain variant that lacks residue 1 (desB1). In yet other instances, the A chain may be a RLN2 A chain variant that lacks residues 1 to 4 (desA1-4) and the B chain may be a RLN2 B chain variant that lacks residue 1 (desB1). In certain instances, the A chain is the desA1-4 variant. In certain instances, the B chain is the desB1 variant.

Other A and B chains that can be used in the RLN analogs herein are described in, for example, Intl. Patent Application Publication Nos. WO 2018/148419, WO 2018/138170, WO 2017/201340, WO 2016/149501, WO 2015/157829, WO 2015/067791, WO 2015/067113, WO 2014/102179, WO 2013/177529, WO 2013/007563, WO 2013/004607, WO 2012/031326 and WO 2012/024452; and US Patent Application Publication No. US 2011/0243942. See also, Chan et al. (2012) J. Biol. Chem. 287:41152-41164; Claasz et al. (2002) Eur. J. Biochem. 269:6287-6293; Hossain et al. (2015) Org. Biomol. Chem. 13:10895-10890; Hossain et al. (2016) Chem. Sci. 7:3805-3819; Park et al. (2008) J Biol. Chem. 283:32099-32109 and Wilkinson et al. (2005) BMC Evol. Biol. 5:14.

With regard to L₁, it can be a peptide of about 1 amino acid to about 50 amino acids. Alternatively, L₁ can be from about 1, about 5, about 10, about 15, about 20, about 25, about 30, about 35, about 40, about 45 or about 50 amino acids. Alternatively still, L₁ can be from about 5 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids. In some instances, L₁ may be omitted such that the A chain or B chain is directly conjugated to the VHH moiety. In some instances, L₁ can include a repeating sequence of (GGGGQ)ₙ (SEQ ID NO:14), where n can be from about 1 to about 10, especially 5 (i.e., (GGGGQ)₅; SEQ ID NO: 19). In other instances, L₁ can include a repeating sequence of (PGPQ)ₙ (SEQ ID NO:17), where n can be from about 1 to about 10, especially 8 (i.e., (PGPQ)₈; SEQ ID NO:20). In still other instances, L₁ can include a repeating sequence of (PGPA)ₙ (SEQ ID NO:18), where n can be from about 1 to about 10, especially 8 (i.e., (PGPA)₈; SEQ ID NO:21).

Other linkers that can be used in the RLN analogs as L₁ include, but are not limited to, (GGGQ)ₙ (SEQ ID NO:15) or (GGGGS)ₙ (SEQ ID NO:16).

With regard to L₂, it can be a peptide of about 1 amino acid to about 15 amino acids. Alternatively, L₂ can be about 1, about 2, about 3, about 4, about 5, about 6, about 7, about 8, about 9, about 10, about 11, about 12, about 13, about 14, or about 15 amino acids. Alternatively still, L₂ can be about 1 amino acid to about 5 amino acids, about 5 amino acids to about 10 amino acids, about 10 amino acids to about 15 amino acids, especially 10 amino acids to 15 amino acids. In some instances, L₂ can include a mix of Ala/A, Gln/Q, Gly/G, Pro/P and Ser/S residues. In other instances, L₂ can be SEQ ID NO:22, 23 or 67.

With regard to VHH, it can be a polypeptide of about 50 amino acids to about 200 amino acids, especially about 125 amino acids to about 150 amino acids that can bind serum albumin or another serum protein having a long t½. In some instances, VHH can be any one of SEQ ID NOS:10 to 13. Alternatively, VHH can be any one of SEQ ID NOS:45-66. The structural features of these VHH moieties result in RLN analogs having a longer t½ when compared to a native RLN, especially native, human RLN2 (SEQ ID NOS:5 and 6). Given that the VHH moieties herein target serum albumin, the t½ of the RLN analog therefore can be expected to be similar to that of serum albumin of the species to which the RLN analog is administered (taking into account any target mediated drug disposition).

In addition to the changes described in the disclosure, the RLN analogs herein may include one or more additional amino acid modifications, especially conservative substitutions, provided, however, that the RLN analogs remain capable of binding to and activating the RXFP1 receptor.

Taken together, exemplary RLN analogs are as follows:
RLN Analog 1, which includes a VHH moiety (underlined), a (G4Q)₅ L₁ (italicized), an A chain of RLN2, a L₂ of ten residues (bolded) and a B chain (desB1) of RLN2, has the following amino acid sequence: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITSKV ADLYPYWGOGTLVTVSSGGGGQGGGGQGGGGQGGGGQGGGGQQLYSALANK CCHVGCTKRSLARFCGGGSGGSGGGSWMEEVIKLCGRELVRAQIAICGMSTWS (SEQ ID NO:24), or a pharmaceutically acceptable salt thereof;
RLN Analog 2, which includes a VHH moiety (underlined), a (PGPA)₈ L₁ (italicized) an A chain of RLN2, a L₂ of ten residues (bolded) and a B chain (desB1) of RLN2, has the following amino acid sequence: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSSPGPAPGPAPGPAPGPAPGPAPGPAPGPAPGPAQLYSAL ANKCCHVGCTKRSLARFCGGGSGGSGGGSWMEEVIKLCGRELVRAQIAICGMS TWS (SEQ ID NO:25), or a pharmaceutically acceptable salt thereof;
RLN Analog 3, which includes a VHH moiety (underlined), a (G4Q)₅ L₁ (italicized), a B chain (desB1) of RLN2, a L₂ of ten residues (bolded) and an A chain of RLN2, has the following amino acid sequence: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSSGGGGQGGGGQGGGGQGGGGQGGGGQSWMEEVIKL CGRELVRAQIAICGMSTWSGGGSGGSGGGQLYSALANKCCHVGCTKRSLARFC (SEQ ID NO:26), or a pharmaceutically acceptable salt thereof;
RLN Analog 4, which includes a VHH moiety (underlined), a (PGPQ)₈ L₁ (italicized), a B chain (desB1) of RLN2, a L₂ of ten residues (bolded) and an A chain of RLN2, has the following amino acid sequence: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSSPGPQPGPQPGPQPGPQPGPQPGPQPGPQPGPQSWME EVIKLCGRELVRAQIAICGMSTWSGGGSGGSGGGQLYSALANKCCHVGCTKRS LARFC (SEQ ID NO:27), or a pharmaceutically acceptable salt thereof;
RLN Analog 5, which includes a B chain (desB1) of RLN2, a L₂ of ten residues (bolded), an A chain of RLN2, a (G4Q)₅ L₁ (italicized) and a VHH moiety (underlined), has the following amino acid sequence: SWMEEVIKLCGRELVRAQIAICGMSTWSGGGSGGSGGGQLYSALANKCCHVGC TKRSLARFCGGGGQGGGGQGGGGQGGGGQGGGGQEVQLLESGGGLVQPGGSL RLSCAASGRYIDETAVAWFRQAPGKEREFVAGIGGGVDITYYADSVKGRFTISRD NSKNTLYLQMNSLRPEDTAVYYCAARPGRPLITSKVADLYPYWGQGTLVTVSSP P (SEQ ID NO:28), or a pharmaceutically acceptable salt thereof;
RLN Analog 6, which includes a B chain (desB1) of RLN2, a L₂ of ten residues (bolded), an A chain of RLN2, a (PGPQ)₅ L₁ (italicized) and a VHH moiety (underlined), has the following amino acid sequence: SWMEEVIKLCGRELVRAQIAICGMSTWSGGGSGGSGGGQLYSALANKCCHVGC TKRSLARFCPGPQPGPQPGPQPGPQPGPQPGPQPGPQPGPQEVQLLESGGGLVQ PGGSLRLSCAASGRYIDETAVAWFRQAPGKEREFVAGIGGGVDITYYADSVKGR FTISRDNSKNTLYLQMNSLRPEDTAVYYCAARPGRPLITSKVADLYPYWGQGTL VTVSSPP (SEQ ID NO:29), or a pharmaceutically acceptable salt thereof;
RLN Analog 7, which includes a VHH moiety (underlined), a (G4Q)₅ L₁ (italicized), a B chain (desB1) of RLN2, a L₂ of ten residues (bolded) and an A chain of RLN2, has the following amino acids sequence: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV ADLYPYWGQGTLVTVSSGGGGQGGGGQGGGGQGGGGQGGGGQSWMEEVIKL CGRELVRAQIAICGMSTWSGGGSGGSGGGQLYSALANKCCHVGCTKRSLARFC (SEQ ID NO:30), or a pharmaceutically acceptable salt thereof;
RLN Analog 8, which includes a VHH moiety (underlined), a (PGPQ)₈-L₁ (italicized), a B chain (desB1) of RLN2, a L₂ of ten residues (bolded) and an A chain of RLN2, has the following amino acid sequence: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV ADLYPYWGQGTLVTVSSPGPQPGPQPGPQPGPQPGPQPGPQPGPQPGPQSWME EVIKLCGRELVRAQIAICGMSTWSGGGSGGSGGGQLYSALANKCCHVGCTKRS LARFC (SEQ ID NO:31), or a pharmaceutically acceptable salt thereof;
RLN Analog 9, which includes a B chain (desB1) of RLN2, a L₂ of ten residues (bolded), an A chain of RLN2, a (G4Q)₅ L₁ (italicized) and a VHH moiety (underlined), has the following amino acid sequence: SWMEEVIKLCGRELVRAQIAICGMSTWSGGGSGGSGGGQLYSALANKCCHVGC TKRSLARFCGGGGQGGGGQGGGGQGGGGQGGGGQEVQLLESGGGLVQPGGSL RLSCAASGRYIDETAVAWFRQAPGKGREFVAGIGGGVDITYYADSVKGRFTISRD NSKNTLYLQMNSLRPEDTAVYYCAARPGRPLITSKVADLYPYWGQGTLVTVSSP P (SEQ ID NO:32), or a pharmaceutically acceptable salt thereof;
RLN Analog 10, which includes a B chain (desB1) of RLN2, a L₂ of ten residues (bolded), an A chain of RLN2, a (PGPQ)₈ L₁ (italicized) and a VHH moiety (underlined), has the following amino acid sequence: SWMEEVIKLCGRELVRAQIAICGMSTWSGGGSGGSGGGQLYSALANKCCHVGC TKRSLARFCPGPQPGPQPGPQPGPQPGPQPGPQPGPQPGPQEVQLLESGGGLVQ PGGSLRLSCAASGRYIDETAVAWFRQAPGKGREFVAGIGGGVDITYYADSVKGR FTISRDNSKNTLYLQMNSLRPEDTAVYYCAARPGRPLITSKVADLYPYWGQGTL VTVSSPP (SEQ ID NO:33), or a pharmaceutically acceptable salt thereof;
RLN Analog 11, which includes a VHH moiety (underlined), a (G4Q)₅ L₁ (italicized), a B chain (desB1) of RLN2, a L₂ of thirteen residues (bolded) and an A chain (desA1-4) of RLN2, has the following amino acid sequence: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSSGGGGQGGGGQGGGGQGGGGQGGGGQSWMEEVI KLCGRELVRAQIAICGMSTWSGGGSGGSGGSGGGALANKCCHVGCTKRSLARF C (SEQ ID NO:34), or a pharmaceutically acceptable salt thereof;
RLN Analog 12, which includes a VHH moiety (underlined), a (G4Q)₅ L₁ (italicized), a B chain (desB1) of RLN2, a L₂ of thirteen residues (bolded) and an A chain (desA1-4) of RLN2, has the following amino acid sequence: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV ADLYPYWGQGTLVTVSSGGGGQGGGGQGGGGQGGGGQGGGGQSWMEEVIKL CGRELVRAQIAICGMSTWSGGGSGGSGGSGGGALANKCCHVGCTKRSLARFC (SEQ ID NO:35), or a pharmaceutically acceptable salt thereof;
RLN Analog 13, which includes a VHH moiety (underlined), a (G4Q)₅ L₁ (italicized), an A chain (desA1-4) of RLN2, a L₂ of ten residues (bolded) and a B chain (desB1) of RLN2, has the following amino acid sequence: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV ADLYPYWGQGTLVTVSSGGGGQGGGGQGGGGQGGGGQGGGGQALANKCCHV GCTKRSLARFCGGGSGGSGGGSWMEEVIKLCGRELVRAQIAICGMSTWS (SEQ ID NO:36), or a pharmaceutically acceptable salt thereof;
RLN Analog 14, which includes a VHH moiety (underlined), a (PGPQ)₈ L₁ (italicized), an A chain (desA1-4) of RLN2, a L₂ of ten residues (bolded) and a B chain (desB1) of RLN2, has the following amino acid sequence: EVQLLESGGGLVQPGGSLRLSCAASGRYIDETAVAWFRQAPGKGREFVAGIGGG VDITYYADSVKGRFTISRDNSKNTLYLQMNSLRPEDTAVYYCAARPGRPLITSKV ADLYPYWGQGTLVTVSSPGPQPGPQPGPQPGPQPGPQPGPQPGPQPGPQALAN KCCHVGCTKRSLARFCGGGSGGSGGGSWMEEVIKLCGRELVRAQIAICGMSTW S (SEQ ID NO:37), or a pharmaceutically acceptable salt thereof;
RLN Analog 15, which includes a VHH moiety (underlined), a (G4Q)₅ L₁ (italicized), a B chain (native) of RLN2, a L₂ of ten residues (bolded) and an A chain (native) of RLN2, has the following amino acid sequence: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSSGGGGQGGGGQGGGGQGGGGQGGGGQDSWMEEVIK LCGRELVRAQIAICGMSTWSSGGGGSGGGGQLYSALANKCCHVGCTKRSLARF C (SEQ ID NO:38), or a pharmaceutically acceptable salt thereof; and
RLN Analog 16, which includes a VHH moiety (underlined), a (PGPA)₈ L₁ (italicized), a B chain (native) of RLN2, a L₂ of ten residues (bolded) and an A chain (native) of RLN2, has the following amino acid sequence: EVQLLESGGGLVQPGGSLRLSCAASGRYIDETAVAWFRQAPGKEREFVAGIGGG VDITYYADSVKGRFTISRDNSKNTLYLQMNSLRPEDTAVYYCAARPGRPLITSKV ADLYPYWGQGTLVTVSSPGPAPGPAPGPAPGPAPGPAPGPAPGPAPGPADSWME EVIKLCGRELVRAQIAICGMSTWSSGGGGSGGGGQLYSALANKCCHVGCTKRS LARFC (SEQ ID NO:39), or a pharmaceutically acceptable salt thereof.

Half-life of the RLN analogs herein may be measured using methods known in the art including, for example, those described in the Examples below. Likewise, affinity of the RLN analogs for albumins of different species may be measured using methods known in the art for measuring binding affinities, for example, those described in the Examples below, and is commonly expressed as the equilibrium dissociation constant (K_{D}) value. Moreover, activity of the RLN analogs at each of the RXFP receptors may be measured using methods known in the art, including, for example, the in vitro activity assays described below, and is commonly expressed as an EC₅₀ value.

As a result of the modifications described above, the RLN analogs herein have a t½ that is longer than that of a native RLN, especially native, human RLN2 (SEQ ID NOS:5 and 6) when administered to a mammal, especially a human. As noted above, the VHH moieties herein target serum album; therefore, the t½ of the RLN analogs herein can be expected to be similar to that of serum albumin of the species to which the RLN analog is administered. In some instances, the RLN analogs can have a t½ of about 1 day to about 31 days, of about 5 days to about 25 days, of about 10 days to about 20 days, or even of about 15 days. In other instances, the RLN analogs can have a t½ of about 1 day to about 5 days, of about 6 days to about 10 days, of about 11 days to about 15 days, of about 16 days to about 20 days, of about 21 to about 25 days, or even of about 26 to about 31 days. In other instances, the RLN analogs can have a t½ of about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or even about 31 days. In particular instances, the RLN analogs can have a t½ of about 20 days when administered to a human.

Likewise, the RLN analogs herein have a potency at the RXFP1 receptor within about 10-fold to about 100-fold of, for example, native, human RLN2 (SEQ ID NOS:5 and 6) when administered to a human.

### Pharmaceutical Compositions and Kits

The RLN analogs herein can be formulated as pharmaceutical compositions, which can be administered by parenteral routes (e.g., intravenous, intraperitoneal, intramuscular, subcutaneous or transdermal). Such pharmaceutical compositions and techniques for preparing the same are well known in the art. See, e.g., Remington, “The Science and Practice of Pharmacy” (D. B. Troy ed., 21^{st} Ed., Lippincott, Williams & Wilkins, 2006). In particular instances, the RLN analogs are administered SQ or IV. Alternatively, however, the RLN analogs can be formulated in forms for other pharmaceutically acceptable routes such as, for example, tablets or other solids for oral administration; time release capsules, and any other form currently used, including creams, lotions, inhalants and the like.

As noted above, and to improve their in vivo compatibility and effectiveness, the RLN analogs herein may be reacted with any number of inorganic and organic acids/bases to form pharmaceutically acceptable acid/base addition salts. Pharmaceutically acceptable salts and common techniques for preparing them are well known in the art (see, e.g., Stahl et al., “Handbook of Pharmaceutical Salts: Properties, Selection and Use” (2^{nd} Revised Ed. Wiley-VCH, 2011)). Pharmaceutically acceptable salts for use herein include sodium, trifluoroacetate, hydrochloride and acetate salts.

The RLN analogs herein may be administered by a physician or self-administered using an injection. It is understood the gauge size and amount of injection volume can be readily determined by one of skill in the art. However, the amount of injection volume can be ≤about 2 mL or even ≤about 1 mL, and the needle gauge can be ≥about 27 G or even ≥about 29 G.

The disclosure also provides and therefore encompasses novel intermediates and methods useful for synthesizing the RLN analogs herein, or a pharmaceutically acceptable salt thereof. The intermediates and RLN analogs can be prepared by a variety of techniques that are well known in the art. For example, a method using recombinant synthesis is illustrated in the Examples below. The specific steps for each of the techniques described may be combined in different ways to prepare the RLN analogs. The reagents and starting materials are readily available to one of skill in the art.

The RLN analogs herein are generally effective over a wide dosage range. Exemplary doses of the RLN analogs or of pharmaceutical compositions including the same can be milligram (mg) or microgram (μg), nanogram (ng), or picogram (pg) amounts per kilogram (kg) of an individual. In this manner, a daily dose can be from about 1 μg to about 100 mg.

Here, the effective amount of the RLN analog in a pharmaceutical composition can be a dose of about 0.25 mg to about 5.0 mg. One of skill in the art, however, understands that in some instances the effective amount (i.e., dose/dosage) may be below the lower limit of the aforesaid range and be more than adequate, while in other cases the effective amount may be a larger dose and may be employed with acceptable side effects.

In addition to the RLN analog herein, the pharmaceutical composition also can include at least one additional therapeutic agent, especially a therapeutic agent typically used as the standard of care in cardiovascular, pulmonary and renal conditions, diseases and disorders.

In this manner, a pharmaceutical composition can include an effective amount of at least one RLN analog herein, a pharmaceutically acceptable carrier and optionally at least one additional therapeutic agent. For example, the pharmaceutical composition can include an effective amount of a RLN analog of SEQ ID NO:24 and a pharmaceutically acceptable carrier, an effective amount of a RLN analog of SEQ ID NO:25 and a pharmaceutically acceptable carrier, an effective amount of a RLN analog of SEQ ID NO:26 and a pharmaceutically acceptable carrier, an effective amount of a RLN analog of SEQ ID NO:27 and a pharmaceutically acceptable carrier, an effective amount of a RLN analog of SEQ ID NO:28 and a pharmaceutically acceptable carrier, an effective amount of a RLN analog of SEQ ID NO:29 and a pharmaceutically acceptable carrier, an effective amount of a RLN analog of SEQ ID NO:30 and a pharmaceutically acceptable carrier, an effective amount of a RLN analog of SEQ ID NO:31 and a pharmaceutically acceptable carrier, an effective amount of a RLN analog of SEQ ID NO:32 and a pharmaceutically acceptable carrier, an effective amount of a RLN analog of SEQ ID NO:33 and a pharmaceutically acceptable carrier, an effective amount of a RLN analog of SEQ ID NO:34 and a pharmaceutically acceptable carrier, an effective amount of a RLN analog of SEQ ID NO:35 and a pharmaceutically acceptable carrier, an effective amount of a RLN analog of SEQ ID NO:36 and a pharmaceutically acceptable carrier, an effective amount of a RLN analog of SEQ ID NO:37 and a pharmaceutically acceptable carrier, an effective amount of a RLN analog of SEQ ID NO:38 and a pharmaceutically acceptable carrier, or an effective amount of a RLN analog of SEQ ID NO:39 and a pharmaceutically acceptable carrier.

Alternatively, the RLN analogs herein can be provided as part of a kit. In some instances, the kit includes a device for administering at least one RLN analog (and optionally at least one additional therapeutic agent) to an individual. In certain instances, the kit includes a syringe and needle for administering the at least one RLN analog (and optionally at least one additional therapeutic agent). In particular instances, the RLN analog (and optionally at least one additional therapeutic agent) is pre-formulated in aqueous solution within the syringe.

### Methods of Making and Using the RLN Analogs

The RLN analogs herein can be made via any number of standard recombinant DNA methods or standard chemical peptide synthesis methods known in the art. With regard to recombinant DNA methods, one can use standard recombinant techniques to construct a polynucleotide having a nucleic acid sequence that encodes an amino acid sequence for a RLN analog, incorporate that polynucleotide into recombinant expression vectors, and introduce the vectors into host cells, such as bacteria, yeast and mammalian cells, to produce the RLN analog. See, e.g., Green & Sambrook, “Molecular Cloning: A Laboratory Manual” (Cold Spring Harbor Laboratory Press, 4^{th} ed. 2012).

With regard to recombinant DNA methods, the compounds herein can be prepared by producing a protein or precursor protein molecule using recombinant DNA techniques. DNA, including cDNA and synthetic DNA, may be double-stranded or single-stranded, and the coding sequences therein encoding a compound herein may vary as a result of the redundancy or degeneracy of the genetic code. Briefly, the DNA sequences encoding the compounds herein are introduced into a host cell to produce the compound or precursor thereof. The host cells can be bacterial cells such as K12 or B strains of Escherichia coli, fungal cells such as yeast cells, or mammalian cells such as Chinese hamster ovary (CHO) cells.

An appropriate host cell is transiently or stably transfected or transformed with an expression system, such as expression vectors, for producing a compound herein or a precursor thereof. Expression vectors typically are replicable in the host organisms either as episomes or as an integral part of the host chromosomal DNA. Commonly, expression vectors will contain selection markers such as, for example, tetracycline, neomycin, G418 and dihydrofolate reductase, to permit selection of those cells transformed with the desired DNA sequences.

The specific biosynthetic or synthetic steps for each of the steps described herein may be used, not used or combined in different ways to prepare the compounds herein.

With regard to chemical peptide synthesis methods, one can use standard manual or automated solid-phase synthesis procedures. For example, automated peptide synthesizers are commercially available from, for example, Applied Biosystems (Foster City, CA) and Protein Technologies Inc. (Tucson, AZ). Reagents for solid-phase synthesis are readily available from commercial sources. Solid-phase synthesizers can be used according to the manufacturer's instructions for blocking interfering groups, protecting amino acids during reaction, coupling, deprotecting and capping of unreacted amino acids. Additional details on making synthetic RLNs can be found in U.S. Pat. Nos. 4,835,251 and 5,166,191.

One use of the RLN analogs herein is for treating cardiovascular conditions, diseases and/or disorders. Exemplary cardiovascular conditions, diseases and disorders include, but are not limited to, acute heart failure, chronic heart failure, atherosclerosis, coronary artery disease, diabetes, stroke, hypercholesterolemia, hypertension, ischemia, vasoconstriction and ventricular hypertrophy.

Another use of the RLN analogs herein is for treating pulmonary conditions, diseases and/or disorders. Exemplary pulmonary conditions, diseases and disorders include, but are not limited to, pulmonary hypertension and chronic obstructive pulmonary disease (COPD).

Another use of the RLN analogs herein is for treating renal conditions, diseases and/or disorders. Exemplary renal conditions, diseases and disorders include, but are not limited to, chronic kidney disease and diabetes nephropathy.

The methods can include the steps described herein, and these maybe be, but not necessarily, carried out in the sequence as described. Other sequences, however, also are conceivable. Moreover, individual or multiple steps may be carried out either in parallel and/or overlapping in time and/or individually or in multiply repeated steps. Furthermore, the methods may include additional, unspecified steps.

Such methods therefore can include selecting an individual having a cardiovascular condition, disease or disorder or who is predisposed to the same. Alternatively, the methods can include selecting an individual having a pulmonary condition, disease or disorder or who is predisposed to the same. Alternatively, the methods can include selecting an individual having a renal condition, disease or disorder or who is predisposed to the same. In certain instances, the methods can include selecting an individual who is diabetic, hypertensive with kidney function impairment and/or obese.

The methods also can include administering to the individual an effective amount of at least one RLN analog herein, which may be in the form of a pharmaceutical composition as also described herein. In some instances, the RLN analog/pharmaceutical composition can include an additional therapeutic agents such as an anticoagulant, an ACE inhibitor, an ARB, an ARNI, a β-blocker, a diuretic, digitalis, digoxin, hydralazine/isorbide dinitrate, a MRA or other aldosterone antagonist, a SGLT2 inhibitor, a statin and/or an anti-glycemic agent, as well as other therapeutic agents to control comorbidities, including, but not limited to, high cholesterol, high blood pressure, atrial fibrillation and diabetes.

The concentration/dose/dosage of the RLN analog and optional additional therapeutic agent are discussed elsewhere herein.

With regard to a route of administration, the RLN analog or pharmaceutical composition including the same can be administered in accord with known methods such as, for example, orally; by injection (i.e., intra-arterially, intravenously, intraperitoneally, intracerebrally, intracerebroventricularly, intramuscularly, intraocularly, intraportally or intralesionally); by sustained release systems, or by implantation devices. In certain instances, the RLN analog or pharmaceutical composition including the same can be administered SQ by bolus injection or continuously.

With regard to a dosing frequency, the RLN analog or pharmaceutical composition including the same can be administered daily, every other day, three times a week, two times a week, one time a week (i.e., weekly), biweekly (i.e., every other week), or monthly. In certain instances, the RLN analog or pharmaceutical composition including the same is administered SQ every other day, SQ three times a week, SQ two times a week, SQ one time a week, SQ every other week or SQ monthly. In particular instances, the RLN analog or pharmaceutical composition including the same is administered SQ one time a week (QW).

With regard to those instances in which the RLN analog or pharmaceutical composition including the same is administered in combination with an effective amount of at least one additional therapeutic agent. The additional therapeutic agent can be administered simultaneously, separately or sequentially with the RLN analog or pharmaceutical composition including the same.

Moreover, the additional therapeutic agent can be administered with a frequency same as the RLN analog or pharmaceutical composition including the same (i.e., every other day, twice a week, or even weekly). Alternatively, the additional therapeutic agent can be administered with a frequency distinct from the RLN analog or pharmaceutical composition including the same. In other instances, the additional therapeutic agent can be administered SQ. In other instances, the additional therapeutic agent can be administered IV. In still other instances, the additional therapeutic agent can be administered orally.

It is further contemplated that the methods may be combined with diet and exercise and/or may be combined with additional therapeutic agents other than those discussed above.

### EXAMPLES

The following non-limiting examples are offered for purposes of illustration, not limitation.

### Polypeptide Expression

### Example 1: Recombinant Expression of RLN Analog 1

Example 1 is a RLN analog having an amino acid sequence of:

(SEQ ID NO: 24)
EVQLLESGGGLVQPGGSLRLSCAASGRYIDETAVAWFRQAPGKEREFVA
GIGGGVDITYYADSVKGRFTISRDNSKNTLYLQMNSLRPEDTAVYYCAA
RPGRPLITSKVADLYPYWGQGTLVTVSSGGGGQGGGGQGGGGQGGGGQG
GGGQQLYSALANKCCHVGCTKRSLARFCGGGSGGSGGGSWMEEVIKLCG
RELVRAQIAICGMSTWS.

Here, the RLN analog of SEQ ID NO:24 is generated in a mammalian cell expression system using CHOK1 cell derivatives. A cDNA sequence encoding SEQ ID NO:24 is sub-cloned into GS-containing expression plasmid backbone (pEE12.4-based plasmids). The cDNA sequence is fused in frame with the coding sequence of a signal peptide sequence, METDTLLLWVLLLWVPGSTG (SEQ ID NO:44), to enhance secretion of the RLN analog into the tissue culture medium. The expression is driven by the viral CMV promoter.

For generating the RLN analog via transient transfection, CHOK1 cells are transfected with the recombinant expression plasmid using a PEI-based method. Briefly, the appropriate volume of CHOK1 suspension cells at a density of 4×10⁶ cells/mL is transferred in shake flasks, and both PEI and recombinant plasmid DNA are added to the cells. Cells are incubated in a suspension culture at 32° C. for 6 days. At the end of the incubation period, cells are removed by low speed centrifugation and the RLN analog protein is purified from the conditioned medium.

Alternatively, and for generating the RLN analog via stable transfections, CHOK1 cells are stably transfected using electroporation and the appropriate amount of recombinant expression plasmid, and the transfected cells are maintained in suspension culture at an adequate cell density. Selection of the transfected cells is accomplished by growth in 25 μM MSX-containing serum-free medium and incubated at about 35° C.-37° C. and about 5%-7% CO₂.

The RLN analog is secreted into the media from the CHO cells, which is purified by Protein A affinity chromatography followed by ion exchange and hydrophobic interaction chromatography or size-exclusion chromatography. Specifically, the RLN analog from harvested media is captured onto Mab Select Protein A resin (GE). The resin then is briefly washed with a running buffer, such as a phosphate-buffered saline (PBS; pH 7.4) or a buffer containing Tris, to remove non-specifically bound material. The protein is eluted from the resin with a low pH solution, such as 10 mM citric acid pH 3. Fractions containing the RLN analog are pooled and may be held at a low pH to inactivate potential viruses. The pH may be neutralized by adding a base such as 0.1 M Tris pH 8.0. The RLN analog may be further purified by ion exchange chromatography using resins such as Poros 50 HS (ThermoFisher). The RLN analog is eluted from the column using a 0 to 500 mM NaCl gradient in 20 mM NaOAc, pH 5.0 over 15 column volumes.

The RLN analog may be further purified by hydrophobic interaction chromatography by using a Capto Phenyl ImpRes HIC Column (GE Healthcare). The purification is performed by adjusting the column charge solution to around 0.5 M sodium sulfate and eluting using a 10 CV gradient going from 0.5 M to 0 M sodium sulfate in a 20 mM Tris pH 8 solution. After HIC, the RLN analog may be even further purified by SEC by loading the concentrated Capto Phenyl ImpRes pool on a Superdex200 (GE Healthcare) with isocratic elution in PBS pH 7.4 or in 20 mM histidine, 50 mM NaCl pH 6.0.

Purified RLN analog may be passed through a viral retention filter such as Planova 20N (Asahi Kasei Medical) followed by concentration/diafiltration into 20 mM histidine, 20 mM NaCl pH 6 using tangential flow ultrafiltration on a regenerated cellulose membrane (Millipore).

The RLN analog therefore is prepared in this manner or in a similar manner that would be readily determined by one of skill in the art.

### Example 2: Recombinant Expression of RLN Analog 2

Example 2 is a RLN analog having an amino acid sequence of

(SEQ ID NO: 25)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSPGPAPGPAP
GPAPGPAPGPAPGPAPGPAPGPAQLYSALANKCCHVGCTKRSLAR
FCGGGSGGSGGGSWMEEVIKLCGRELVRAQIAICGMSTWS.

Here, the RLN analog of SEQ ID NO:25 is generated essentially as described for Example 1 except that a cDNA sequence encoding SEQ ID NO:25 is used in the expression plasmid.

### Example 3: Recombinant Expression of RLN Analog 3

Example 3 is a RLN analog having an amino acid sequence of:

(SEQ ID NO: 26)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQSWMEEVIKLCGRELVRAQIAICGMSTWSG
GGSGGSGGGQLYSALANKCCHVGCTKRSLARFC.

Here, the RLN analog of SEQ ID NO:26 is generated essentially as described for Example 1 except that a cDNA sequence encoding SEQ ID NO:26 is used in the expression plasmid.

### Example 4: Recombinant Expression of RLN Analog 4

Example 4 is a RLN analog having an amino acid sequence of:

(SEQ ID NO: 27)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSPGPQPGPQP
GPQPGPQPGPQPGPQPGPQPGPQSWMEEVIKLCGRELVRAQIAIC
GMSTWSGGGSGGSGGGQLYSALANKCCHVGCTKRSLARFC.

Here, the RLN analog of SEQ ID NO:27 is generated essentially as described for Example 1 except that a cDNA sequence encoding SEQ ID NO:27 is used in the expression plasmid.

### Example 5: Recombinant Expression of RLN Analog 5

Example 5 is a RLN analog having an amino acid sequence of:

(SEQ ID NO: 28)
SWMEEVIKLCGRELVRAQIAICGMSTWSGGGSGGSGGGQLYSALA
NKCCHVGCTKRSLARFCGGGGQGGGGQGGGGQGGGGQGGGGQEVQ
LLESGGGLVQPGGSLRLSCAASGRYIDETAVAWFRQAPGKEREFV
AGIGGGVDITYYADSVKGRFTISRDNSKNTLYLQMNSLRPEDTAV
YYCAARPGRPLITSKVADLYPYWGQGTLVTVSSPP.

Here, the RLN analog of SEQ ID NO:28 is generated essentially as described for Example 1 except that a cDNA sequence encoding SEQ ID NO:28 is used in the expression plasmid.

### Example 6: Recombinant Expression of RLN Analog 6

Example 6 is a RLN analog having an amino acid sequence of:

(SEQ ID NO: 29)
SWMEEVIKLCGRELVRAQIAICGMSTWSGGGSGGSGGGQLYSALA
NKCCHVGCTKRSLARFCPGPQPGPQPGPQPGPQPGPQPGPQPGPQ
PGPQEVQLLESGGGLVQPGGSLRLSCAASGRYIDETAVAWFRQAP
GKEREFVAGIGGGVDITYYADSVKGRFTISRDNSKNTLYLQMNSL
RPEDTAVYYCAARPGRPLITSKVADLYPYWGQGTLVTVSSPP.

Here, the RLN analog of SEQ ID NO:29 is generated essentially as described for Example 1 except that a cDNA sequence encoding SEQ ID NO:29 is used in the expression plasmid.

### Example 7: Recombinant Expression of RLN Analog 7

Example 7 is a RLN analog having an amino acid sequence of:

(SEQ DI NO: 30)
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQSWMEEVIKLCGRELVRAQIAICGMSTWSG
GGSGGSGGGQLYSALANKCCHVGCTKRSLARFC.

Here, the RLN analog of SEQ ID NO:30 is generated essentially as described for Example 1 except that a cDNA sequence encoding SEQ ID NO:30 is used in the expression plasmid.

### Example 8: Recombinant Expression of RLN Analog 8

Example 8 is a RLN analog having an amino acid sequence of:

(SEQ ID NO: 31)
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKVADLYPYWGQGTLVTVSSPGPQPGPQP
GPQPGPQPGPQPGPQPGPQPGPQSWMEEVIKLCGRELVRAQIAIC
GMSTWSGGGSGGSGGGQLYSALANKCCHVGCTKRSLARFC.

Here, the RLN analog of SEQ ID NO:31 is generated essentially as described for Example 1 except that a cDNA sequence encoding SEQ ID NO:31 is used in the expression plasmid.

### Example 9: Recombinant Expression of RLN Analog 9

Example 9 is a RLN analog having an amino acid sequence of:

(SEQ ID NO: 32)
SWMEEVIKLCGRELVRAQIAICGMSTWSGGGSGGSGGGQLYSALA
NKCCHVGCTKRSLARFCGGGGQGGGGQGGGGQGGGGQGGGGQEVQ
LLESGGGLVQPGGSLRLSCAASGRYIDETAVAWFRQAPGKGREFV
AGIGGGVDITYYADSVKGRFTISRDNSKNTLYLQMNSLRPEDTAV
YYCAARPGRPLITSKVADLYPYWGQGTLVTVSSPP.

Here, the RLN analog of SEQ ID NO:32 is generated essentially as described for Example 1 except that a cDNA sequence encoding SEQ ID NO:32 is used in the expression plasmid.

### Example 10: Recombinant Expression of RLN Analog 10

Example 10 is a RLN analog having an amino acid sequence of

(SEQ ID NO: 33)
SWMEEVIKLCGRELVRAQIAICGMSTWSGGGSGGSGGGQLYSALA
NKCCHVGCTKRSLARFCPGPQPGPQPGPQPGPQPGPQPGPQPGPQ
PGPQEVQLLESGGGLVQPGGSLRLSCAASGRYIDETAVAWFRQAP
GKGREFVAGIGGGVDITYYADSVKGRFTISRDNSKNTLYLQMNSL
RPEDTAVYYCAARPGRPLITSKVADLYPYWGQGTLVTVSSPP.

Here, the RLN analog of SEQ ID NO:33 is generated essentially as described for Example 1 except that a cDNA sequence encoding SEQ ID NO:33 is used in the expression plasmid.

### Example 11: Recombinant Expression of RLN Analog 11

Example 11 is a RLN analog having an amino acid sequence of

(SEQ ID NO: 34)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQSWMEEVIKLCGRELVRAQIAICGMSTWSG
GGSGGSGGSGGGALANKCCHVGCTKRSLARFC.

Here, the RLN analog of SEQ ID NO:34 is generated essentially as described for Example 1 except that a cDNA sequence encoding SEQ ID NO:34 is used in the expression plasmid.

### Example 12: Recombinant Expression of RLN Analog 12

Example 12 is a RLN analog having an amino acid sequence of

(SEQ ID NO: 35)
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQSWMEEVIKLCGRELVRAQIAICGMSTWSG
GGSGGSGGSGGGALANKCCHVGCTKRSLARFC.

Here, the RLN analog of SEQ ID NO:35 is generated essentially as described for Example 1 except that a cDNA sequence encoding SEQ ID NO:35 is used in the expression plasmid.

### Example 13: Recombinant Expression of RLN Analog 13

Example 13 is a RLN analog having an amino acid sequence of

(SEQ ID NO: 36)
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQALANKCCHVGCTKRSLARFCGGGSGGSGG
GSWMEEVIKLCGRELVRAQIAICGMSTWS.

Here, the RLN analog of SEQ ID NO:36 is generated essentially as described for Example 1 except that a cDNA sequence encoding SEQ ID NO:36 is used in the expression plasmid.

### Example 14: Recombinant Expression of RLN Analog 14

Example 14 is a RLN analog having an amino acid sequence of:

(SEQ ID NO: 37)
EVQLLESGGGLVQPGGSLRLSCAASGRYIDETAVAWFRQAPGKGR
EFVAGIGGGVDITYYADSVKGRFTISRDNSKNTLYLQMNSLRPED
TAVYYCAARPGRPLITSKVADLYPYWGQGTLVTVSSPGPQPGPQP
GPQPGPQPGPQPGPQPGPQPGPQALANKCCHVGCTKRSLARFCGG
GSGGSGGGSWMEEVIKLCGRELVRAQIAICGMSTWS.

Here, the RLN analog of SEQ ID NO:37 is generated essentially as described for Example 1 except that a cDNA sequence encoding SEQ ID NO:37 is used in the expression plasmid.

### Example 15: Recombinant Expression of RLN Analog 15

Example 15 is a RLN analog having an amino sequence of:

(SEQ ID NO: 38)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSGGGGQGGGG
QGGGGQGGGGQGGGGQDSWMEEVIKLCGRELVRAQIAICGMSTWS
SGGGGSGGGGQLYSALANKCCHVGCTKRSLARFC.

Here, the RLN analog of SEQ ID NO:38 is generated essentially as described for Example 1 except that a cDNA sequence encoding SEQ ID NO:38 is used in the expression plasmid.

### Example 16: Recombinant Expression of RLN Analog 16

Example 16 is a RLN analog having an amino sequence of:

(SEQ ID NO: 39)
EVQLLESGGGLVQPGGSLRLSCAASGRYIDETAVAWFRQAPGKER
EFVAGIGGGVDITYYADSVKGRFTISRDNSKNTLYLQMNSLRPED
TAVYYCAARPGRPLITSKVADLYPYWGQGTLVTVSSPGPAPGPAP
GPAPGPAPGPAPGPAPGPAPGPADSWMEEVIKLCGRELVRAQIAI
CGMSTWSSGGGGSGGGGQLYSALANKCCHVGCTKRSLARFC.

Here, the RLN analog of SEQ ID NO:39 is generated essentially as described for Example 1 except that a cDNA sequence encoding SEQ ID NO:39 is used in the expression plasmid.

### In Vitro Function

### Example 17: RLN Analog Albumin-Binding Via SPR

In vitro binding of a RLN analog to human, cynomolgus monkey, mouse, rat, pig, dog, cow and rabbit serum albumin is determined by SPR. In particular, the affinity of the RLN analogs of Examples 1 to 14 to serum albumin of these species is summarized below in Tables 1 to 14.

Binding of the RLN analogs of Examples 1 to 14 to various serum albumins is carried out on Biacore 8K instrument. The immobilization of the serum albumin to a Series S Sensor Chip CM5 surface is performed according to the manufacturer's instructions (Amine Coupling Kit BR-1000-50). Briefly, carboxyl groups on the sensor chip surfaces (flow cell 1 and 2) are activated by injecting 70 μL of a mixture containing 75 mg/mL EDC and 11.5 mg/mL NHS at 10 μL/min. Human, cynomolgus monkey, mouse, rat, pig, dog, cow and rabbit serum albumin are diluted in 10 mM sodium acetate pH 4.0 (BR-1003-49) at 0.5, 0.5, 2.2, 0.6, 0.6, 0.8, 0.6 and 0.3 g/mL and then injected over the activated chip surfaces (flow cell 2, channel 1 to 8) at 10 μL/min for 180 sec (human, mouse, rat, pig and cow serum albumin are obtained from Sigma Aldrich (St. Louis, MO); cynomolgus monkey serum albumin is obtained from Hölzel Diagnostika (Cologne, Germany); dog serum albumin is obtained from Molecular Innovations (Novi, MI); and rabbit serum albumin is obtained from Fitzgerald Industries Intl. (Acton, MA). The various serum albumins are covalently immobilized through free amines onto a carboxymethyl dextran-coated sensor chip CM5 targeting a surface density of about 100 (62-145) RU. Excess reactive groups on the surfaces (flow cell 1 and 2) are deactivated by injecting 70 μL of 1 M ETA HCl—NaOH pH 8.5.

The RLN analogs of Examples 1 to 14 are diluted in HBS-EP+ buffer (10 mM HEPES pH 7.6, 150 mM NaCl, 3 mM EDTA, 0.05% Polysorbate 20) at concentrations of 1000, 333.3, 111.1, 37.04, 12.35, 4.12, 1.37, 0.457, 0.152, 0.051 and 0.017 nM. 150 μl of sample is individually injecting sequentially across the immobilized serum albumins on the chip's surface and dissociating for 600 sec at 50 μL/min flow rate at 25° C. The surface is regenerated by injecting 10 mM glycine-HCl pH 1.5 (BR-1003-54) at 50 μL/min for 100 sec. The resulting sensorgrams are analyzed using Biacore 8K Insight Evaluation Software (version 2.0.15.12933) 1:1 binding kinetics model fitting to calculate the binding kinetic parameter association rate (ka), dissociation rate (kd), and equilibrium dissociation constant (K_{D}).

TABLE 1
Binding Kinetics of RLN Analog of Example 1
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 9.8E+05 | 1.1E−04 | 1.1E−10 |
| Cyno SA | 1.1E+06 | 5.3E−04 | 5.0E−10 |
| Mouse SA | 1.3E+06 | 4.2E−03 | 3.3E−09 |
| Rat SA | 1.2E+06 | 2.6E−03 | 2.1E−09 |
| Pig SA | 7.8E+05 | 5.5E−03 | 7.1E−09 |
| Dog SA | 1.3E+06 | 1.5E−03 | 1.2E−09 |
| Cow SA | 1.4E+06 | 5.0E−02 | 3.7E−08 |

| | |
|---|---|
| Rabbit SA | No binding |

K_{D} is determined as 0.1, 0.5, 3.3, 2.1, 7.1, 1.2 and 37 nM for human, cynomolgus monkey, mouse, rat, pig, dog and cow serum albumin binding, respectively, with the RLN analog of Example 1.

TABLE 2
Binding Kinetics of RLN Analog of Example 2
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 9.2E+05 | 9.5E−05 | 1.0E−10 |
| Cyno SA | 1.2E+06 | 5.2E−04 | 4.5E−10 |
| Mouse SA | 1.1E+06 | 4.2E−03 | 3.8E−09 |
| Rat SA | 9.8E+05 | 2.9E−03 | 3.0E−09 |
| Pig SA | 6.9E+05 | 5.6E−03 | 8.0E−09 |
| Dog SA | 1.0E+06 | 1.5E−03 | 1.4E−09 |
| Cow SA | 9.8E+05 | 4.9E−02 | 5.0E−08 |

| | |
|---|---|
| Rabbit SA | No binding |

K_{D} is determined as 0.1, 0.45, 3.8, 3.0, 8.0, 1.4 and 50 nM for human, cynomolgus monkey, mouse, rat, pig, dog and cow serum albumin binding, respectively, with the RLN analog of Example 2.

TABLE 3
Binding Kinetics of RLN Analog of Example 3
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 1.4E+06 | 1.1E−04 | 7.6E−11 |
| Cyno SA | 1.5E+06 | 5.4E−04 | 3.6E−10 |
| Mouse SA | 1.7E+06 | 4.0E−03 | 2.4E−09 |
| Rat SA | 1.7E+06 | 2.7E−03 | 1.6E−09 |
| Pig SA | 1.1E+06 | 5.3E−03 | 4.8E−09 |
| Dog SA | 2.0E+06 | 1.4E−03 | 7.0E−10 |

| | | |
|---|---|---|
| Cow SA | n/a (steady state) | 2.6E−08 |

| | |
|---|---|
| Rabbit SA | No binding |

K_{D} is determined as 0.08, 0.36, 2.4, 1.6, 4.8, 0.7 and 26 nM for human, cynomolgus monkey, mouse, rat, pig, dog and cow serum albumin binding, respectively, with the RLN analog of Example 3.

TABLE 4
Binding Kinetics of RLN Analog of Example 4
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 1.2E+06 | 1.1E−04 | 8.9E−11 |
| Cyno SA | 1.3E+06 | 5.3E−04 | 4.2E−10 |
| Mouse SA | 1.3E+06 | 3.9E−03 | 3.0E−09 |
| Rat SA | 1.2E+06 | 2.6E−03 | 2.1E−09 |
| Pig SA | 8.6E+05 | 5.3E−03 | 6.2E−09 |
| Dog SA | 1.4E+06 | 1.4E−03 | 1.0E−09 |

| | | |
|---|---|---|
| Cow SA | n/a (steady state) | 3.7E−08 |

| | |
|---|---|
| Rabbit SA | No binding |

K_{D} is determined as 0.09, 0.42, 3.0, 2.1, 6.2, 1.0 and 37 nM for human, cynomolgus monkey, mouse, rat, pig, dog and cow serum albumin binding, respectively, with the RLN analog of Example 4.

TABLE 5
Binding Kinetics of RLN Analog of Example 5
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 5.8E+05 | 2.6E−04 | 4.5E−10 |
| Cyno SA | 8.2E+05 | 1.5E−03 | 1.8E−09 |
| Mouse SA | 5.9E+05 | 8.3E−03 | 1.4E−08 |
| Rat SA | 7.0E+05 | 7.2E−03 | 1.0E−08 |
| Pig SA | 5.8E+05 | 1.3E−02 | 2.3E−08 |
| Dog SA | 7.1E+05 | 3.4E−03 | 4.7E−09 |

| | | |
|---|---|---|
| Cow SA | n/a (steady state) | 1.2E−07 |

| | |
|---|---|
| Rabbit SA | No binding |

K_{D} is determined as 0.45, 1.8, 14, 10, 23, 4.7 and 120 nM for human, cynomolgus monkey, mouse, rat, pig, dog and cow serum albumin binding, respectively, with the RLN analog of Example 5.

TABLE 6
Binding Kinetics of RLN Analog of Example 6
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 4.7E+05 | 2.4E−04 | 5.1E−10 |
| Cyno SA | 4.3E+05 | 1.0E−03 | 2.3E−09 |
| Mouse SA | 6.3E+05 | 7.6E−03 | 1.2E−08 |
| Rat SA | 4.9E+05 | 4.8E−03 | 9.7E−09 |
| Pig SA | 4.1E+05 | 9.9E−03 | 2.4E−08 |
| Dog SA | 4.8E+05 | 2.0E−03 | 4.1E−09 |

| | | |
|---|---|---|
| Cow SA | n/a (steady state) | 1.3E−07 |

| | |
|---|---|
| Rabbit SA | No binding |

K_{D} is determined as 0.51, 2.3, 12, 9.7, 24, 4.1 and 130 nM for human, cynomolgus monkey, mouse, rat, pig, dog and cow serum albumin binding, respectively, with the RLN analog of Example 6.

TABLE 7
Binding Kinetics of RLN Analog of Example 7
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 1.9E+06 | 1.2E−04 | 6.3E−11 |
| Cyno SA | 2.1E+06 | 5.9E−04 | 2.9E−10 |
| Mouse SA | 2.0E+06 | 4.1E−03 | 2.1E−09 |
| Rat SA | 2.0E+06 | 2.8E−03 | 1.4E−09 |
| Pig SA | 1.5E+06 | 5.4E−03 | 3.7E−09 |
| Dog SA | 2.2E+06 | 1.5E−03 | 6.9E−10 |

| | | |
|---|---|---|
| Cow SA | n/a (steady state) | 2.5E−08 |

| | |
|---|---|
| Rabbit SA | No binding |

K_{D} is determined as 0.06, 0.29, 2.1, 1.4, 3.7, 0.69 and 25 nM for human, cynomolgus monkey, mouse, rat, pig, dog and cow serum albumin binding, respectively, with the RLN analog of Example 7.

TABLE 8
Binding Kinetics of RLN Analog of Example 8
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 1.5E+06 | 1.1E−04 | 7.8E−11 |
| Cyno SA | 1.9E+06 | 5.9E−04 | 3.2E−10 |
| Mouse SA | 2.2E+06 | 3.9E−03 | 1.8E−09 |
| Rat SA | 1.6E+06 | 2.8E−03 | 1.7E−09 |
| Pig SA | 1.3E+06 | 5.2E−03 | 4.1E−09 |
| Dog SA | 2.3E+06 | 1.5E−03 | 6.6E−10 |

| | | |
|---|---|---|
| Cow SA | n/a (steady state) | 2.1E−08 |

| | |
|---|---|
| Rabbit SA | No binding |

K_{D} is determined as 0.08, 0.32, 1.8, 1.7, 4.1, 0.66 and 21 nM for human, cynomolgus monkey, mouse, rat, pig, dog and cow serum albumin binding, respectively, with the RLN analog of Example 8.

TABLE 9
Binding Kinetics of RLN Analog of Example 9
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 9.5E+05 | 1.8E−04 | 1.9E−10 |
| Cyno SA | 1.1E+06 | 9.4E−04 | 8.6E−10 |
| Mouse SA | 1.2E+06 | 1.1E−02 | 9.5E−09 |
| Rat SA | 9.1E+05 | 7.5E−03 | 8.2E−09 |
| Pig SA | 8.7E+05 | 1.2E−02 | 1.4E−08 |
| Dog SA | 9.5E+05 | 3.6E−03 | 3.8E−09 |

| | | |
|---|---|---|
| Cow SA | n/a (steady state) | 8.4E−08 |

| | |
|---|---|
| Rabbit SA | No binding |

K_{D} is determined as 0.19, 0.86, 9.5, 8.2, 14, 3.8 and 84 nM for human, cynomolgus monkey, mouse, rat, pig, dog and cow serum albumin binding, respectively, with the RLN analog of Example 9.

TABLE 10
Binding Kinetics of RLN Analog of Example 10
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 5.5E+05 | 2.0E−04 | 3.7E−10 |
| Cyno SA | 5.1E+05 | 7.7E−04 | 1.5E−09 |
| Mouse SA | 7.4E+05 | 7.2E−03 | 9.7E−09 |
| Rat SA | 6.3E+05 | 4.9E−03 | 7.8E−09 |
| Pig SA | 4.9E+05 | 9.6E−03 | 2.0E−08 |
| Dog SA | 6.3E+05 | 2.1E−03 | 3.4E−09 |

| | | |
|---|---|---|
| Cow SA | n/a (steady state) | 1.0E−07 |

| | |
|---|---|
| Rabbit SA | No binding |

K_{D} is determined as 0.37, 1.5, 9.7, 7.8, 20, 3.4 and 100 nM for human, cynomolgus monkey, mouse, rat, pig, dog and cow serum albumin binding, respectively, with the RLN analog of Example 10.

TABLE 11
Binding Kinetics of RLN Analog of Example 11
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 1.2E+06 | 2.1E−04 | 1.7E−10 |
| Cyno SA | 1.3E+06 | 6.4E−04 | 4.8E−10 |
| Mouse SA | 1.4E+06 | 5.0E−03 | 3.6E−09 |
| Rat SA | 2.1E+06 | 2.8E−03 | 1.3E−09 |
| Pig SA | 8.5E+05 | 6.2E−03 | 7.3E−09 |
| Dog SA | 1.4E+06 | 1.7E−03 | 1.3E−09 |

| | | |
|---|---|---|
| Cow SA | n/a (steady state) | 2.7E−08 |

| | |
|---|---|
| Rabbit SA | No binding |

K_{D} is determined as 0.17, 0.48, 3.6, 1.3, 7.3, 1.3 and 27 nM for human, cynomolgus monkey, mouse, rat, pig, dog and cow serum albumin binding, respectively, with the RLN analog of Example 11.

TABLE 12
Binding Kinetics of RLN Analog of Example 12
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 1.4E+06 | 1.9E−04 | 1.4E−10 |
| Cyno SA | 1.5E+06 | 7.0E−04 | 4.7E−10 |
| Mouse SA | 1.8E+06 | 4.9E−03 | 2.6E−09 |
| Rat SA | 2.0E+06 | 3.3E−03 | 1.7E−09 |
| Pig SA | 1.4E+06 | 5.8E−03 | 4.2E−09 |
| Dog SA | 2.1E+06 | 1.6E−03 | 7.7E−10 |

| | | |
|---|---|---|
| Cow SA | n/a (steady state) | 3.0E−08 |

| | |
|---|---|
| Rabbit SA | No binding |

K_{D} is determined as 0.14, 0.47, 2.6, 1.7, 4.2, 0.77 and 30 nM for human, cynomolgus monkey, mouse, rat, pig, dog and cow serum albumin binding, respectively, with the RLN analog of Example 12.

TABLE 13
Binding Kinetics of RLN Analog of Example 13
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 1.4E+06 | 1.9E−04 | 1.4E−10 |
| Cyno SA | 1.4E+06 | 7.0E−04 | 4.9E−10 |
| Mouse SA | 1.8E+06 | 4.8E−03 | 2.6E−09 |
| Rat SA | 1.7E+06 | 3.3E−03 | 2.0E−09 |
| Pig SA | 1.2E+06 | 6.0E−03 | 5.0E−09 |
| Dog SA | 1.9E+06 | 1.7E−03 | 9.2E−10 |

| | | |
|---|---|---|
| Cow SA | n/a (steady state) | 2.9E−08 |

| | |
|---|---|
| Rabbit SA | No binding |

K_{D} is determined as 0.14, 0.49, 2.6, 2.0, 5.0, 0.92 and 29 nM for human, cynomolgus monkey, mouse, rat, pig, dog and cow serum albumin binding, respectively, with the RLN analog of Example 13.

TABLE 14
Binding Kinetics of RLN Analog of Example 14
to Human, Cynomolgus Monkey, Mouse, Rat, Pig,
Dog, Cow and Rabbit Serum Albumin at 25° C.

| Binding to Immobilized | | | |
|---|---|---|---|
| Serum Albumins (SA) | ka (1/Ms) | kd (1/s) | K_{D} (M) |
| Human SA | 1.2E+06 | 1.9E−04 | 1.6E−10 |
| Cyno SA | 1.1E+06 | 6.8E−04 | 6.1E−10 |
| Mouse SA | 1.5E+06 | 4.6E−03 | 3.1E−09 |
| Rat SA | 1.4E+06 | 3.2E−03 | 2.2E−09 |
| Pig SA | 9.7E+05 | 5.8E−03 | 6.0E−09 |
| Dog SA | 1.5E+06 | 1.7E−03 | 1.1E−09 |

| | | |
|---|---|---|
| Cow SA | n/a (steady state) | 3.4E−08 |

| | |
|---|---|
| Rabbit SA | No binding |

K_{D} is determined as 0.16, 0.61, 3.1, 2.2, 6.0, 1.1 and 34 nM for human, cynomolgus monkey, mouse, rat, pig, dog and cow serum albumin binding, respectively, with the RLN analog of Example 14.

### Example 18: RLN Analog In Vitro Potency at RXFP1 and RXFP2 Receptors

Generating CHO-CRE-Luc cell line: CHO-K1 cells (ATCC) are cultured in DMEM-F12 3:1 with 5% FBS with 20 mM HEPES, 40 μg/mL L-proline, 1× antibiotics and split 1:5 every 2-3 days with TrypLE™ Express (Gibco). Cells are transfected with plasmid DNA of pGL4.29[luc2P/CRE/Hygro](Promega) and Fugene HD (Promega) according to the manufacturer's instructions. Transfected cells are selected with hygromycin B at 1 mg/mL for 3-4 weeks. Clonal lines are obtained by limited dilution cloning into 96-well plates and are confirmed with a forskolin response by luciferase assay with Bright-Glo Reagent (Promega). Clones are expanded, harvested, resuspended in freezing media, aliquoted into cryovials, and kept in liquid nitrogen for long-term storage. The top responder is selected with the best forskolin response (signal to background ratio), clonal line #2B6, for subsequent transfection with human RXFP1 and RXFP2 receptors.

Generating CHO human RXFP1- and human RXFP2-expressing cell lines: CHO-CRE-Luc line #2B6 cells are cultured in DMEM-F12 3:1 with 5% FBS with 20 mM HEPES, 40 μg/mL L-proline, 1× antibiotics, 1 mg/mL hygromycin B and split 1:5 every 2-3 days with TrypLE Express (Gibco). Cells are transfected with plasmid DNA of human RXFP1 receptor or human RXFP2 receptor and Fugene HD (Promega) according to the manufacturer's instructions. Transfected cells are selected with hygromycin B (1 mg/mL) and puromycin (6 μg/mL) for 3-4 weeks. Clonal lines are obtained by limited dilution cloning into 96-well plates. Clonal lines are confirmed by a human RLN2 response. Clones are expanded, harvested, resuspended in freezing media, aliquoted into cryovials, and kept frozen under liquid nitrogen for long-term storage. Clonal lines are selected with the best response to human RLN2 (signal to background ratio) for assay validation.

Human RXFP1 and RXFP2 receptor luciferase assay: CHO cell lines expressing the human RXFP1 or the human RXFP2 are cultured with selection medium (DMEM-F12 3:1 with 5% FBS with 20 mM HEPES, 40 μg/mL L-proline, 1× antibiotics, 6 μg/mL puromycin, 1 mg/mL hygromycin B). On Day −1 (the day before cAMP CRE luciferase reporter assay), cells are washed once with PBS, lifted from flasks with cell dissociation solution (enzyme free cell dissociation solution, GIBCO cat #13151-014:TrypLE™ Express=30:1), and resuspended in plating medium (DMEM-F12 3:1 with 20 mM HEPES, 1× antibiotics, 0.5% FBS). Cells are plated in a 96-well plate (Falcon Cat #353219) at 20,000 cells/0.1 mL/well. Cells are cultured at 37° C. 5% CO₂ overnight. On Day 1 (the day of cAMP CRE luciferase reporter assay), medium is removed and replaced with 90 μL serum-free medium (DMEM-F12 3:1 with 20 mM HEPES, 1× antibiotics). Plates are incubated at 37° C. for 2 hr, then 10 μL of 10× ligand is added (RLN2, final 1×). Plates are incubated for an additional 4 hr at 37° C. After the incubation period is complete, plates are brought to room temperature for 15 min. Then, 50 μL of Bright-Glo™ is added to each well, and plates are read on a Biotek Neo2 reader with Gen5 software.

Statistical analysis of data: Data is imported from the Biotek Neo2 reader into GraphPad Prism® software (GraphPad Software, LLC; La Jolla, CA; version 7). EC₅₀ values are generated by a variable slope-four parameter dose response curve analysis.

TABLE 15
In Vitro Potency of RLN Analogs to RXPF1 and RXPF2 Receptors.

| | SEQ | hRXFP1 | | | hRXFP2 | | | |
|---|---|---|---|---|---|---|---|---|
| | ID | EC₅₀ nM | | | EC₅₀ nM | | | R2/R1 |
| Compound | NO: | GeoMean | SEM | N | GeoMean | SEM | N | ratio |
| hRLN2 | 5 & 6 | 0.15 | 0.012 | 4 | 1.7 | 0.45 | 8 | 11 |
| Example 1 | 24 | 3.0 | 2.8 | 6 | 65 | 17 | 2 | 22 |
| Example 2 | 25 | 1.1 | 0.64 | 6 | 79 | 6.2 | 2 | 74 |
| Example 3 | 26 | 4.0 | 1.0 | 2 | 19 | 2.4 | 4 | 5 |
| Example 4 | 27 | 2.9 | 0.2 | 2 | 24 | 6.0 | 4 | 8 |
| Example 5 | 28 | 8.1 | 2.9 | 2 | 98 | 16 | 4 | 12 |
| Example 6 | 29 | 15 | 3.0 | 2 | 39 | 4.9 | 4 | 3 |
| Example 7 | 30 | 0.91 | 0.04 | 2 | 26 | 2.5 | 4 | 28 |
| Example 8 | 31 | 2.0 | 1.2 | 2 | 32 | 14 | 4 | 16 |
| Example 9 | 32 | 5.2 | 0.01 | 2 | 97 | 14 | 4 | 19 |
| Example 10 | 33 | 14 | 0.33 | 2 | 32 | 3.9 | 4 | 2 |
| Example 11 | 34 | 3.2 | 0.69 | 2 | 207 | 31 | 4 | 65 |
| Example 12 | 35 | 2.9 | 0.51 | 2 | 193 | 29 | 4 | 66 |
| Example 13 | 36 | 10 | 0.8 | 2 | 407 | 17 | 3 | 42 |
| Example 14 | 37 | 17 | 2.3 | 2 | 628 | 51 | 3 | 37 |
| Example 15 | 38 | 1.7 | 0.13 | 5 | 23 | 3.6 | 4 | 14 |
| Example 16 | 39 | 2.0 | 0.22 | 4 | 70 | 23 | 2 | 35 |

### In Vivo Function

### Example 19: Pharmacokinetics of RLN Analogs in Male Sprague Dawley Rats

Male Sprague Dawley rats are administered a single SQ dose of 200 nmol/kg of a RLN analog in His-NaCl buffer (pH 6.0) at a volume of 1.0 mL/kg. Blood is collected 3, 6, 12, 24, 48, 72, 96, 120, 144, 168 and 240 hr post-dose for pharmacokinetic characterization.

Plasma concentrations of the RLN analogs are determined by a qualified LC/MS method at Eli Lilly and Company. The Example compounds and an analog internal standard are extracted from 100% rat plasma using a human RLN antibody followed by detecting an N-terminal tryptic peptide using a Q-Exactive™ Orbitrap® mass spectrometer.

Data for the RLN analogs of Examples 4 and 7 are provided below in Table 16.

TABLE 16
Mean Plasma Pharmacokinetic Parameters for RLN Analogs Following
a Single 200 nmol/kg SQ Dose to Male Sprague Dawley Rats.

| | Animal | t½ | Tₘₐₓ | Cₘₐₓ | AUC_{0-inf} | CL/F |
|---|---|---|---|---|---|---|
| Compound | ID | (hr) | (hr) | (nmol/L) | (hr*nmol/L) | (mL/hr/kg) |
| Example 4 | 1 | 28.0 | 72 | 297 | 35510 | 5.63 |
| | 2 | 28.8 | 48 | 368 | 37529 | 5.33 |
| | 3 | 32.8 | 48 | 356 | 39220 | 5.10 |
| | Mean | 29.9 | 56 | 340 | 37419 | 5.35 |
| | SD | 2.6 | 14 | 38 | 1857 | 0.27 |
| Example 7 | 1 | 40.1 | 48 | 387 | 51939 | 3.85 |
| | 2 | 37.9 | 72 | 487 | 57843 | 3.46 |
| | 3 | 31.3 | 48 | 344 | 44409 | 4.50 |
| | Mean | 36.4 | 56 | 406 | 51397 | 3.94 |
| | SD | 4.6 | 14 | 74 | 6734 | 0.53 |

NOTE:
Abbreviations: t½ = half-life, Tₘₐₓ = time to maximum concentration, Cₘₐₓ = maximum observed plasma concentration, AUC_{0-inf} = area under the curve from time 0 hours to infinity, CL/F = clearance/bioavailability, N = 3 animals/group/time point.
As shown in Table 16, the RLN analogs of Examples 4 and 7 demonstrate an extended pharmacokinetic profile in Sprague Dawley rats.

### Example 20: In Vivo Effect of RLN Analogs on Renal Blood Flow Following IV Administration in Male Sprague Dawley Rats

Male, five-week old Sprague Dawley rats (Charles River Laboratories, Inc.) are housed in a vivarium on a normal light/dark cycle for one week prior to start of the experiment. The rats then are randomized into the following treatment groups: vehicle (20 mM His/20 mM NaCl, pH 6.0 buffer) and the RLN analog of Example 7 based on body weight. The RLN analog is dosed at 2.44 μg/kg IV bolus followed by 0.36 μg/kg/hr IV infusion, 8.13 μg/kg IV bolus followed by 1.2 μg/kg/hr IV infusion, 24.4 μg/kg IV bolus followed by 3.6 μg/kg/hr IV infusion, and 81.3 μg/kg IV bolus followed by 11.9 μg/kg/hr IV infusion.

To measure the effect of the RLN analog on renal blood flow, rats are anesthetized with urethane (1.2 g/kg, IP) and are prepared for abdominal/renal ultrasound imaging and renal artery pulsed-wave Doppler blood flow measurements (VisualSonics, Model Vevo 3100 ultrasound system; Fujifilm). A chronic tail vein catheter is placed for the IV bolus and infusion administration. After a 30-min acclimation period, a baseline and 3-hr post-start of dose renal blood flow measurements are acquired.

TABLE 17
Effect of IV Administered RLN Analog on Renal Blood Flow in Healthy Rats.

| | | | Percent Change |
|---|---|---|---|
| | | Renal Blood Flow | from Vehicle |
| Compound and Dose | Time | (mL/min) | (%) |
| Vehicle at 1.6 mL/kg IV bolus | baseline | 8.58 ± 0.16 | |
| then 0.475 mL/kg/hr IV infusion | 3-hr | 8.56 ± 0.18 | — |
| Example 7 at 2.44 μg/kg IV bolus | baseline | 8.49 ± 0.31 | |
| then 0.36 μg/kg/hr IV infusion | 3-hr | 8.59 ± 0.33 | 0.4 |
| Example 7 at 8.13 μg/kg IV bolus | baseline | 8.63 ± 0.44 | |
| then 1.2 μg/kg/hr IV infusion | 3-hr | 10.84 ± 0.53†* | 26.3 |
| Example 7 at 24.4 μg/kg IV bolus | baseline | 8.54 ± 0.23 | |
| then 3.6 μg/kg/hr IV infusion | 3-hr | 12.74 ± 0.37†* | 48.8 |
| Example 7 at 81.3 μg/kg IV bolus | baseline | 8.34 ± 0.15 | |
| then 11.9 μg/kg/hr IV infusion | 3-hr | 13.73 ± 0.47†* | 60.4 |

NOTE:
results expressed as Mean ± SD, †significantly different from baseline p < 0.001 ANOVA,
*significantly different from vehicle p < 0.001 ANOVA, N = 5 per treatment group.

As seen in Table 17, the RLN analog of Example 7 significantly increases renal blood flow in the 8.13 μg/kg IV bolus followed by 1.2 μg/kg/hr IV infusion, 24.4 μg/kg IV bolus followed by 3.6 μg/kg/hr IV infusion, and 81.3 μg/kg IV bolus followed by 11.9 μg/kg/hr IV infusion treated groups by 26.6, 48.8 and 60.4% after 3 hr of exposure.

### Example 21: In Vivo Effect of RLN Analogs on Renal Blood Flow Following SQ Administration in Male Sprague Dawley Rats

Male, five-week old Sprague Dawley rats (Charles River Laboratories, Inc.) are housed in a vivarium on a normal light/dark cycle for one week prior to start of the experiment. The rats then are randomized into the following treatment groups: vehicle (20 mM His/20 mM NaCl, pH 6.0 Buffer) and the RLN Analog of Example 7 based on body weight. The RLN analog is dosed SQ at 180 μg/kg.

To measure the effect of the RLN analog on renal blood flow, 48 hr after dosing the rats are anesthetized with urethane (1.2 g/kg, IP) and are prepared for abdominal/renal ultrasound imaging and renal artery pulsed-wave Doppler blood flow measurements (VisualSonics, Model Vevo 3100 ultrasound system; Fujifilm). After a 30-min acclimation period, the renal blood flow measurements are acquired.

TABLE 18
Effect of SQ Administered RLN Analog
on Renal Blood Flow in Healthy Rats.

| | | Percent Change |
|---|---|---|
| | Renal Blood Flow | from Vehicle |
| Compound and Dose | (mL/min) | (%) |
| Vehicle at 5 mL/kg | 8.51 ± 0.33 | — |
| Example 7 at 180 μg/kg | 11.27 ± 0.81* | 32.4 |

NOTE:
results expressed as Mean ± SD, *significantly different from vehicle p < 0.001 ANOVA, N = 10 per treatment group.

As seen in Table 18, the RLN analog of Example 7 significantly increases renal blood flow after 48 hr of exposure to a SQ dose of 180 μg/kg.

The invention claimed is:

1. A compound comprising a structure of: 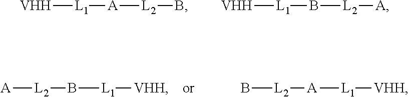
wherein VHH comprises an amino acid sequence selected from the group consisting of SEQ ID NOS:10, 11, 12 and 13 or a sequence having at least 90% sequence similarity thereto,
wherein A is a relaxin A chain comprising an amino acid sequence selected from the group consisting of SEQ ID NOS:2, 5 and 8 or a sequence having at least 90% sequence similarity thereto,
wherein B is a relaxin B chain comprising an amino acid sequence selected from the group consisting of SEQ ID NOS:3, 6 and 9 or a sequence having at least 90% sequence similarity thereto,
wherein L₁ is a first linker comprising an amino acid sequence selected from the group consisting of (GGGGQ)ₙ (SEQ ID NO:14), (PGPQ)ₙ (SEQ ID NO:17) and (PGPA)ₙ (SEQ ID NO:18), and wherein n can be from 1 to 10, and
wherein L₂ is a second linker comprising an amino acid sequence selected from the group consisting of SEQ ID NOS:22, 23 and 67; or
a pharmaceutically acceptable salt thereof.

2. The compound of claim 1, wherein A is SEQ ID NO:2.

3. The compound of claim 1, wherein B is SEQ ID NO:3.

4. The compound of claim 1, wherein A is SEQ ID NO:5.

5. The compound of claim 1, wherein B is SEQ ID NO:6.

6. The compound of claim 1, wherein A is SEQ ID NO:5 and lacks the first four amino acids (desA1-4).

7. The compound of claim 1, wherein B is SEQ ID NO:6 and lacks the first amino acid (desB1).

8. The compound of claim 1, wherein A is SEQ ID NO:5 and lacks the first four amino acids (desA1-4), and wherein B is SEQ ID NO:6 and lacks the first amino acid (desB1).

9. The compound of claim 1, wherein A is SEQ ID NO:8.

10. The compound of claim 1, wherein B is SEQ ID NO:9.

11. The compound of claim 1, wherein L₁ is SEQ ID NO:19.

12. The compound of claim 1, wherein L₁ is SEQ ID NO:20.

13. The compound of claim 1, wherein L₁ is SEQ ID NO:21.

14. The compound of claim 1 comprising an amino acid sequence selected from the group consisting of SEQ ID NOS:24 to 39 or a sequence having at least 90% sequence similarity thereto or a pharmaceutically acceptable salt thereof.

15. The compound of claim 1 consisting essentially of an amino acid sequence selected from the group consisting of SEQ ID NOS:24 to 39 or a sequence having at least 90% sequence similarity thereto or a pharmaceutically acceptable salt thereof.

16. The compound of claim 1 consisting of an amino acid sequence selected from the group consisting of SEQ ID NOS:24 to 39 or a sequence having at least 90% sequence similarity thereto or a pharmaceutically acceptable salt thereof.

17. A pharmaceutical composition comprising:
a compound of claim 1 or a pharmaceutically acceptable salt thereof; and
a pharmaceutically acceptable buffer.

18. A method of treating cardiac, pulmonary and/or renal conditions, diseases and/or disorders in an individual, the method comprising the step of:
administering to the individual an effective amount of a compound of claim 1.

19-21. (canceled)

22. A compound comprising an amino acid sequence of selected from the group consisting of SEQ ID NOS: 10-13 and 45-66 or a sequence having at least 90% sequence similarity thereto.

23. The compound of claim 22, comprising an amino acid sequence of SEQ ID NO:11 or a sequence having at least 90% sequence similarity thereto.

24. The compound of claim 22, comprising an amino acid sequence of SEQ ID NO:12 or a sequence having at least 90% sequence similarity thereto.

25. The compound of claim 22, comprising an amino acid sequence of SEQ ID NO:13 or a sequence having at least 90% sequence similarity thereto.

26. The compound of claim 22, comprising an amino acid sequence of SEQ ID NO:45 or a sequence having at least 90% sequence similarity thereto.

27. The compound of claim 22, comprising an amino acid sequence of SEQ ID NO:46 or a sequence having at least 90% sequence similarity thereto.

28. The compound of claim 22, comprising an amino acid sequence of SEQ ID NO:47 or a sequence having at least 90% sequence similarity thereto.

29. The compound of claim 22, comprising an amino acid sequence of SEQ ID NO:48 or a sequence having at least 90% sequence similarity thereto.

30. The compound of claim 22, comprising an amino acid sequence of SEQ ID NO:49 or a sequence having at least 90% sequence similarity thereto.

31. The compound of claim 22, comprising an amino acid sequence of SEQ ID NO:50 or a sequence having at least 90% sequence similarity thereto.

32. The compound of claim 22, comprising an amino acid sequence of SEQ ID NO:51 or a sequence having at least 90% sequence similarity thereto.

33. The compound of claim 22, comprising an amino acid sequence of SEQ ID NO:52 or a sequence having at least 90% sequence similarity thereto.

34. The compound of claim 22, comprising an amino acid sequence of SEQ ID NO:53 or a sequence having at least 90% sequence similarity thereto.

35. The compound of claim 22, comprising an amino acid sequence of SEQ ID NO:54 or a sequence having at least 90% sequence similarity thereto.

36. The compound of claim 22, comprising an amino acid sequence of SEQ ID NO:55 or a sequence having at least 90% sequence similarity thereto.

37. The compound of claim 22, comprising an amino acid sequence of SEQ ID NO:56 or a sequence having at least 90% sequence similarity thereto.

38. The compound of claim 22, comprising an amino acid sequence of SEQ ID NO:57 or a sequence having at least 90% sequence similarity thereto.

39. The compound of claim 22, comprising an amino acid sequence of SEQ ID NO:58 or a sequence having at least 90% sequence similarity thereto.

40. The compound of claim 22, comprising an amino acid sequence of SEQ ID NO:59 or a sequence having at least 90% sequence similarity thereto.

41. The compound of claim 22, comprising an amino acid sequence of SEQ ID NO:60 or a sequence having at least 90% sequence similarity thereto.

42. The compound of claim 22, comprising an amino acid sequence of SEQ ID NO:61 or a sequence having at least 90% sequence similarity thereto.

43. The compound of claim 22, comprising an amino acid sequence of SEQ ID NO:62 or a sequence having at least 90% sequence similarity thereto.

44. The compound of claim 22, comprising an amino acid sequence of SEQ ID NO:63 or a sequence having at least 90% sequence similarity thereto.

45. The compound of claim 22, comprising an amino acid sequence of SEQ ID NO:64 or a sequence having at least 90% sequence similarity thereto.

46. The compound of claim 22, comprising an amino acid sequence of SEQ ID NO:65 or a sequence having at least 90% sequence similarity thereto.

47. The compound of claim 22, comprising an amino acid sequence of SEQ ID NO:66 or a sequence having at least 90% sequence similarity thereto.

48. The compound of claim 22 comprising an amino acid sequence of SEQ ID NO:10 or a sequence having at least 90% sequence similarity thereto.
